(19) United States
(12) Patent Application Publication
(10) Pub. No.: US 2011/0274661 A1
(43) Pub. Date: Nov. 10, 2011

(54) Attentuated Herpesvirus Encoding a Mek Pathway Polypeptide

(75) Inventors: Alice P. Poon, Oakland, CA (US); Ralph R. Weichselbaum, Chicago, IL (US); Bernard Roizman, Chicago, IL (US)

(73) Assignee: The University of Chicago, Chicago, IL (US)

(21) Appl. No.: 13/058,780

(22) PCT Filed: Aug. 7, 2009
(86) PCT No.: PCT/US09/53186

§ 371 Date: Jul. 28, 2011

Related U.S. Application Data
(60) Provisional application No. 61/087,909, filed on Aug. 11, 2008.

(51) Int. Cl. A61K 35/76; A61M 37/00; A61P 19/02; A61P 27/02; C12N 7/04; A61P 35/00

(52) U.S. Cl. 424/93.6; 435/236; 600/1

(57) ABSTRACT

The disclosure provides materials and methods for the treatment of cells exhibiting a cell proliferative disorder with a herpes simplex virus having a deficiency in the expression of active ICP34.5 and comprising an expression control element effective in modulating at least one component of the MEK pathway to ensure that infected cells are MEK+. Cell proliferative diseases, disorders or conditions, such as cancers, rheumatoid arthritis and macular degeneration, are amenable to treatment using these HSVs. Further provided are methods for preventing such cell proliferative disorders by administering the HSVs as well as methods for ameliorating a symptom associated with a cell proliferative disorder by administering such HSVs.

### GOVERNMENT INTEREST

This invention was made with U.S. government support under grant no. CA7193307-07 awarded by the National Institutes of Health. The U.S. government has certain rights in the invention.

### BACKGROUND

In the general field of human health and animal welfare, a variety of diseases, disorders, and conditions have largely eluded the best efforts at prevention or treatment. Chief among such maladies is the loss of cell-cycle control that frequently results in the undesirable cell proliferation characteristic of cancer in its many forms. Malignant gliomas, for example, are devastating brain tumors that afflict animals such as humans. The average life span after diagnosis is less than one year and few patients have been reported to survive five years. Furthermore, none of the conventional anti-cancer therapies has been successful in significantly prolonging the lifespan of patients with this disease. Many of the more devastating forms of cancer, such as malignant gliomas and metastasized forms of a variety of cancers, are inoperable, further reducing the likelihood of receiving effective treatment with conventional therapies.

One approach to the development of new and effective anti-cancer therapies has been directed at engineered viral therapeutics. Chief among the viruses being explored for use as oncolytic agents are genetically engineered forms of herpes simplex viruses (HSV). Because wild-type viruses are highly virulent, the viruses used in preclinical evaluations and in phase-1 clinical studies have been thoroughly attenuated. While several deletion mutants have been tested, the mutants that reached clinical trials lacked a functional γ₁34.5 gene encoding infected cell protein 34.5 (ICP34.5).

In principle, use of an avirulent mutant of herpes simplex viruses 1 (HSV-1) to destroy cancer cells in situ, e.g., in inoperable human tumors, is a sound approach to treating such disease conditions. As noted above, the most promising HSV candidate is an HSV mutant lacking a functional γ₁34.5 gene. The product of the γ₁34.5 gene of HSV, ICP34.5, is a multifunctional protein that blocks a major host response to infection. In brief, after the onset of viral DNA synthesis, infected cells accumulate large amounts of complementary viral RNA transcripts. The consequence of this accumulation is the activation of double-stranded RNA-dependent protein kinase R (PKR). In infected cells, activated PKR phosphorylates the α subunit of the eukaryotic translation initiation factor 2 (eIF-2α), resulting in loss of protein synthesis. In the case of HSV-1, ICP34.5 acts as a phosphatase accessory factor to recruit protein phosphatase 1α to dephosphorylate eIF-2α. As a consequence, protein synthesis continues unimpeded. Mutants derived from Δγ₁34.5 viruses lack the capacity to counteract PKR-induced loss of protein synthesis and cell apoptosis. Another significant property of γ₁34.5 mutant HSV is that they are highly attenuated in animal model systems and phase I clinical studies have demonstrated that Δγ₁34.5 mutants can be administered safely at escalating doses in patients with malignancy. A major impediment to the widespread use of these mutants for cancer therapy is the observation that in animal model systems, human tumor cells differ widely with respect to their ability to support the replication of γ₁134.5 mutant HSV. In cancer cells that do support replication of γ₁34.5-deficient HSV, these viral constructs exhibit lytic cytotoxicity specific to the cancer cells, and are able to act on such cells regardless of body location and distribution. Thus, a need exists in the art for effective and safe viral-based therapies to treat cell proliferation disorders such as cancers.

Investigations of eukaryotic cell physiology have revealed a variety of signal transduction pathways involved in the coordinate regulation of complex physiological processes such as cell proliferation. For example, mitogen-activated protein kinases (MAPKs) have been implicated as elements of regulatory pathways controlling cell proliferation in all eukaryotes. The MAPK pathway is organized in modules, of which there are six different modules presently known. This pathway typically contains an “upstream” (i.e., early step in the pathway) G-protein and a core module containing three kinase enzymes: a MAPK kinase kinase (i.e., MAPKKK) that phosphorylates and thereby activates a MAPK Kinase (i.e., MAPKK), which in turn phosphorylates and activates a MAPK. In one example, the ERK (extracellular-signal-regulated) pathway, Ras is a G-protein, Raf is a MAPKKK, MEK (i.e., MAPK/ERK Kinase) is a MAPKK and ERK is a MAPK. Complicating even this one example of a MAPK signal transduction pathway regulating cell proliferation is the existence of a number of isoforms for the particular kinases. For example, there are three mammalian Raf isoforms, i.e., Raf-1, A-Raf and B-Raf; two MEK isoforms, i.e., MEK1 and MEK2; and two ERK isoforms, i.e., ERK1 and ERK2. Moreover, other kinase enzymes can be substituted for the prototypes listed above. For example, in addition to Raf kinases, MEKK-1, (i.e., MEK Kinase-1), mos or Tp1-2 can activate MEK isoforms.

Complicating the regulatory picture even further, the MAPK pathway also embraces a variety of accessory proteins such as exchange factors, modulators, scaffolding molecules, adapter proteins, and chaperones, collectively providing capacities to localize elements of the pathway, to translocate elements, to finely control the activation/inhibition of elements of the pathway and to ensure that signal propagation is achieved in an efficient and directed manner. An illustrative exchange factor is the Ras GTP/GDP exchange factor known as Son of Sevenless (SOS), a protein that promotes the exchange of GTP for GDP on Ras, thereby activating cell membrane-bound Ras. An example of a modulator involved in the MAPK pathway is SUR-8 (i.e., Suppressor of Ras-8), which binds to Raf-1 and Ras-GTP, forming a ternary complex that enhances Raf-1 activation. Two exemplary scaffolding proteins are the mammalian Kinase Suppressor of Ras (i.e., KSR) and the yeast PBS2 protein (i.e., polymyxin B sensitivity). KSR has been shown to associate with elements of the above-described module of the MAPK pathway, i.e., Raf, MEK and ERK. Consistent with its role as a scaffolding protein for elements of the pathway, KSR has been shown to either activate or inhibit the MAPK pathway, depending on the stoichiometric ratios of KSR to the elements of the pathway (e.g., Raf, MEK, and ERK). In terms of non-binding theory, either an insufficiency or an excess of KSR relative to the pathway components or elements would be expected to lead to an unorganized or poorly organized pathway impeding the capacity of the elements to cooperatively propagate a signal, e.g., a signal modulating cell proliferation. An example of an adapter protein is the mammalian 14-3-3 protein, which modulates a variety of signaling proteins, for example by changing the subcellular location of target proteins or by altering protein associations. As a consequence, 14-3-3 plays a role in regulating cell-cycle checkpoints, cell proliferation, cell differentiation and cell apoptosis. Finally, the MAPK pathway comprehends chaperones such as Hsp90, Hsp50/Cdc37, FKBP65 and Bag-1. Loss of functional chaperone activity results in reduced kinase activity and may be due to a chaperone's stabilization of kinase tertiary structure and/or a role for the chaperone in recruiting kinase, e.g., Raf-1, activators.

The preceding discussion of MAPK pathways illustrates the classes of proteins involved in these complex pathways of regulating such physiological processes as cell proliferation and cell apoptosis. Additional elements of the pathways are known in the art, as illustrated by the disclosures in Kolch, W., J. Biochem. 351:289-305 (2000) and English et al., Exp. Cell Res. 253:255-270 (1999), both of which are incorporated herein by reference in their entireties.

Applications of HSV-1 oncolytic therapy have principally utilized local injection of virus directly into the tumor. For this reason, HSV-1 vectors have been clinically tested primarily in malignant gliomas which remain confined to the CNS. In the context of developing HSV-1 as a broader anticancer agent, it would be valuable to be able to administer HSV-1 systemically (intravenously or intraperitoneally) to effectively treat disseminated metastases in addition to the primary tumor. Metastatic disease is responsible for the vast majority of cancer deaths, often in spite of control of the primary tumor. Moreover, a variety of human tumor types, such as melanomas, sarcomas, and carcinomas of the colon, ovary, liver, breast, esophagus, stomach, pancreas, and lung have been reported to overexpress MEK activity.

Thus, a need continues to exist in the art for virus-based cancer therapeutics and corresponding methods for use in treating a variety of target cancer cells amenable to such virus-based treatment. Accordingly, a need also exists for identifying amenable target cancer cells suitable for virus-based anti-cancer treatment.

### SUMMARY

The invention disclosed herein satisfies at least one of the aforementioned needs in the art by providing therapeutic agents in the form of herpes simplex viruses that do not elaborate wild-type levels of active ICP34.5, the γ₁34.5 gene product. These therapeutic agents are useful in treating target cells exhibiting a cell proliferative disorder, such as a cancer (including a solid-tumor cancer), rheumatoid arthritis, macular degeneration and other diseases, disorders and conditions known in the art to be associated with abnormal, preferably elevated, cell proliferation. Further, such HSVs are shown herein to exhibit improved replication, and hence cytotoxicity due to lytic cell cycle completion, in target cells having an active MAPK pathway, e.g., an active Ras/Rak/MEK/ERK pathway. Delivery of γ₁34.5 deficient HSV, such as R3616, selectively targets and destroys human xenograft tumors that overexpress MEK activity as compared to tumors that express lower MEK activity. In addition, effective delivery can be achieved by a variety of routes, including systemic administration. The results reported herein indicate that systemic delivery of γ₁34.5 deficient HSV is effective in the treatment of human tumors. The invention also provides a method for identifying or diagnosing a cell proliferative disorder amenable to treatment with the above-described HSVs by determining the status of a MAPK pathway in a candidate target cell exhibiting a cell proliferative disorder. Those candidate target cells that have an active MAPK pathway are preferred target cells for administration of the above-described HSVs. In providing methods for advantageously using viral-based therapy for the treatment of cell proliferation diseases, disorders or conditions, the invention provides the benefit of effective treatment for those diseases, disorders or conditions that have proven refractory to conventional treatment, such as inoperable tumors and metastasized cancers. One aspect of the disclosure is drawn to an attenuated herpesvirus expressing less 1CP34.5 activity than a wild-type herpesvirus and an inducible expression control element operatively linked to a coding region for a polypeptide in the MEK pathway. Suitable herpesviruses include HSV-1. Viral attenuation is conveniently achieved by using herpesviruses having one or more γ₁34.5 genes expressing less active ICP34.5 than a wild-type herpesvirus, such as by having a herpesvirus bearing an inactivating mutation (e.g., a deletion) in both copies of the γ₁34.5 gene. Exemplary γ₁34.5 gene deletions involve the deletion of at least 10% of each coding region for ICP34.5 and they γ₁34.5 gene deletions of HSV R3616. In the herpesvirus according to this aspect of the disclosure, any coding region or gene known in the art to be associated with or within the MEK pathway is contemplated for inclusion in the virus. Exemplary coding regions encode a polypeptide in the MEK pathway that is selected from the group consisting of MEK1, MEK2, ERK1, ERK2, Raf-1, A-Raf, B-Raf, mos, Tp1-2, K-Ras, Ras, H-Ras and N-Ras. Other exemplary coding regions encode a polypeptide in the MEK pathway that is selected from the group consisting of K-Ras V12, K-Ras D12. K-Ras G12, H-Ras V12, K-Ras D13, N-Ras V12, Raf S338A, Raf S339A, B-Raf V600E, Raf-CAAX, Raf BXB, ΔN3MKK1 S218E/S222D, ΔN3MKK2 S218E/S222D, ERK2 E58Q, ERK2 D122A, ERK2 S151A, ERK2 S221A, ERK2 S151D ERK L73P and a full-length MEK-ERK fusion. The inducible expression control element of this aspect of the disclosure is inducible by radiation, by exposure to a chemotherapeutic agent, or by any other means known in the art. One type of an inducible expression control element useful in these herpesviruses is a radioinducible promoter. Exemplary radioinducible promoters include promoters selected from the group consisting of an Egr-1 promoter, a c-JUN promoter, a TNF-α promoter, an MDR1 promoter, a tPA promoter, a recA promoter, a p21 (WAF1) promoter, a CMVIE promoter, an SV40 promoter, a pE9 promoter, a survivin promoter, an IEX-1 promoter and a PKC promoter. In one embodiment, the radioinducible promoter is the promoter for HSV gC. Other inducible expression control elements are responsive to at least one chemotherapeutic agents, such as a chemotherapeutic agent selected from the group consisting of (a) an alkylating agent, such as a nitrogen mustard (e.g., mechlorethamine, cylophosphamide, ifosfamide, melphalan, chlorambucil), an ethylenimine or a methylmelamine (e.g., hexamethylmelamine, thiotepa), an alkyl sulfonate (e.g., busulfan), a nitrosourea (e.g., carmustine, Iomustine, chlorozoticin, streptozocin) or a triazine (e.g., dicarbazine); (b) an antimetabolite, such as a folic acid analog (e.g., methotrexate), a pyrimidine analog (e.g., 5-fluorouracil, floxuridine, cytarabine, azauridine) as well as a purine analog or a related compound (e.g., 6-mercaptopurine, 6-thioguanine, pentostatin); (c) a natural product, such as a vinca alkaloid (e.g., vinblastine, vincristine), an epipodophylotoxin (e.g., etoposide, teniposide), an antibiotic (e.g., dactinomycin, daunorubicin, doxorubicin, bleomycin, plicamycin and mitoxanthrone), an enzyme (e.g., L-asparaginase), or a biological response modifier (e.g., Interferon-c0; or (d) a miscellaneous agent, such as a platinum coordination complex (e.g., cisplatin, carboplatin), a substituted urea (e.g., hydroxyurea), a methylhydiazine derivative (e.g., procarbazine), or an adreocortical suppressant (e.g., taxol and mitotane). In some embodiments, cisplatin is a particularly suitable chemotherapeutic agent.

The herpesviruses according to this aspect of the disclosure may further comprise a coding region for an expressible marker, such as the luc coding region encoding the luciferase enzyme. The expressed marker is useful in monitoring the location, or tissue distribution, of viral treatment and/or the quantity of expression exhibited by the therapeutic viruses.

Another aspect according to the disclosure is directed to a method of treating a cell proliferative disorder comprising administering a therapeutically effective amount of a herpesvirus as described herein in combination with a therapeutically effective amount of an anti-cell proliferation agent selected from the group consisting of radiation and a chemotherapeutic agent. Various forms of radiation are contemplated, including proton emission, neutron emission, an a radioisotope, a β radioisotope, a γ radioisotope and ultraviolet radiation. In some embodiments, the therapeutic radiation is ionizing radiation, such as targeted ionizing radiation. Disorders amenable to the method of treatment include, but are not limited to, a cancer, rheumatoid arthritis and macular degeneration.

Yet another aspect according to the disclosure is a method of ameliorating a symptom of a cell proliferative disorder comprising administering a therapeutically effective amount of a herpesvirus as described herein in combination with a therapeutically effective amount of an anti-cell proliferation agent selected from the group consisting of radiation and a chemotherapeutic agent. Forms of radiation and chemotherapeutic agents identified in the context of describing other aspects of the disclosure are suitable for use in the methods of ameliorating symptoms of a cell proliferative disorder.

Another aspect according to the disclosure is a use of the herpesvirus described herein in the preparation of a medicament for the treatment of a subject with a cell proliferation disorder. Still another aspect of the disclosure is a composition comprising the herpesvirus described herein in combination with a pharmaceutically acceptable adjuvant, carrier or diluent.

By way of further illustration, an aspect of the disclosure is drawn to a method of treating a cell proliferation (or cell proliferative) disorder comprising administration of an effective amount of a γ₁34.5 deficient herpes simplex virus, such as a γ₁34.5 deficient herpes simplex virus-1, comprising at least one expressible coding region of the MAPK, or MEK, pathway to a subject in need. In some embodiments, the method comprises administration of a γ₁34.5 deficient herpes simplex virus-1 that comprises a coding region for MEK. In exemplary embodiments, the MEK is selected from the group consisting of MEK1 and MEK2. In some embodiments, the γ₁34.5 deficient herpes simplex virus-1 comprises a coding region for ERK, such as ERK1 or ERK2. In some embodiments, the γ₁34.5 deficient herpes simplex virus-1 comprises a coding region for Raf. In exemplary embodiments, the Raf is selected from the group consisting of Raf-1, A-Raf and B-Raf. In some embodiments, γ₁34.5 deficient herpes simplex virus-1 comprises a coding region for a protein selected from the group consisting of MEK Kinase-1, mos and Tp1-2. Embodiments of the method according to this aspect of the disclosure may comprise administration of a γ₁34.5 deficient herpes simplex virus-1 that comprises a coding region for Ras. In other embodiments of the method according to the disclosure, the coding region for the MAPK pathway encodes a variant of a member of the pathway. In particular embodiments, the variant is selected from the group consisting of K-Ras V12, K-Ras D12, H-Ras V12, K-Ras D13, N-Ras V12, Raf S338A, Raf S339A, B-Raf V600E, Raf-CAAX, Raf BXB, ΔN3MKK1 S218E/S222D, ΔN3MKK2 S218E/S222D, ERK2 E58Q, ERK2 D122A, ERK2 S151A, ERK2 S221A, ERK2 S151D ERK L73P and a full-length MEK-ERK fusion. Other embodiments comprise administration of an effective amount of a γ₁34.5 deficient herpes simplex virus-1 comprising at least one expressible coding region encoding a protein selected from the group consisting of a catalytically inactive mutant of PKR, a catalytically inactive mutant of eIF-2α, a growth factor and an active mutant of a tyrosine kinase receptor, wherein the protein and encoding nucleic acid are known in the art.

In embodiments of this aspect of the disclosure, the γ₁34.5 deficient herpes simplex virus-1 lacks any γ₁34.5 gene. In some embodiments, the γ₁34.5 deficient herpes simplex virus-1 comprises a γ₁34.5 gene with a point mutation. Also contemplated are HSV that are γ₁34.5 deficient due to an inability to effectively express an otherwise intact γ₁34.5 gene. Additionally contemplated are HSV combining the various mechanisms for rendering the virus γ₁34.5 deficient, such as by deletion of one γ₁34.5 gene and mutation of a second γ₁34.5 gene, for example by insertional inactivation, partial deletion, or non-silent point mutation.

The methods according to this aspect of the disclosure extend to methods wherein the treating ameliorates at least one symptom associated with the cell proliferation disorder. Exemplary symptoms include pain, swelling, or loss of physiological function due to cell proliferation, or a tumor mass impinging on one or more tissues or organs.

A variety of cell proliferation, or cell proliferative, disorders are comprehended by the disclosure, including cancer, macular degeneration, and autoimmune disease.

Another aspect of the disclosure is use of a γ₁34.5 deficient HSV comprising at least one expressible coding region of the MAPK pathway in the preparation of a medicament for the treatment of a patient with a cell proliferation disorder. Comprehended in various embodiments of the use are the MAPK pathway coding regions identified above in the context of describing the treatment methods according to the disclosure, i.e., MEK (e.g., MEK1 and/or MEK2), ERK (e.g., ERK1 and/or ER1(2), Raf (e.g., Raf-1, A-Raf and/or B-Raf), Ras, MEK Kinase-1, mos, Tp1-2, variants of each of the members of the MAPK pathway, such as K-Ras V12, K-Ras D12, H-Ras V12, K-Ras D13, N-Ras V12, Raf S338A, Raf S339A, B-Raf V600E, Raf-CAAX, Raf BXB, ΔN3MKK1 S218E/S222D, ΔN3MKK2 S218E/S222D, ERK2 E58Q, ERK2 D122A, ERK2 S151A, ERK2 S221A, ERK2 S151D ERK L73P and a full-length MEK-ERK fusion, and a catalytically inactive mutant of PKR, a catalytically inactive mutant of eIF-2α, a growth factor and an active mutant of a tyrosine kinase receptor. Additionally, the use may comprise any of a variety of γᵢ34.5 deficient HSV, as described herein.

Yet another aspect of the disclosure is a γ₁34.5 deficient HSV comprising at least one expressible coding region of the MAPK pathway. As noted for the aspects of the disclosure described above, the expressible MAPK pathway coding region may be a region encoding MEK (e.g., MEK1 and/or MEK2), ERK (e.g., ERK1 and/or ERK2), Raf (e.g., Raf-1, A-Raf, and B-Raf), Ras, MEK Kinase-1, mos, Tp1-2, variants of each of the members of the MAPK pathway, such as K-Ras V12, K-Ras D12, H-Ras V12, K-Ras D13, N-Ras V12, Raf S338A, Raf S339A, B-Raf V600E, Raf-CAAX, Raf BXB, ΔN3MKK1 S218E/S222D, ΔN3MKK2 S218E/S222D, ERK2 E58Q, ERK2 D122A, ERK2 S151A, ERK2 S221A, ERK2 S151D ERK L73P and a full-length MEK-ERK fusion, and a catalytically inactive mutant of PKR, a catalytically inactive mutant of eIF-2α, a growth factor and an active mutant of a tyrosine kinase receptor. This aspect of the disclosure comprehends a variety of HSV that are γ₁34.5 deficient HSV, such as a γ₁34.5 deficient herpes simplex virus-1 that lacks any γ₁34.5 gene (i.e., an HSV containing a deletion of each of the two γ₁34.5 genes found in wild-type HSV). Further comprehended is a γ₁34.5 deficient herpes simplex virus-1 that comprises a γ₁34.5 gene with a point mutation. Also contemplated are HSV that are γ₁34.5 deficient due to an inability to effectively express an otherwise intact γ₁34.5 gene. Additionally contemplated are HSV combining the various mechanisms for rendering the virus γ₁34.5 deficient, such as by deletion of one γᵢ34.5 gene and mutation of a second γ₁34.5 gene, for example by insertional inactivation, partial deletion, or non-silent point mutation.

A related aspect of the disclosure is drawn to a composition comprising the γ₁34.5 deficient HSV as described above in combination with a pharmaceutically acceptable adjuvant, carrier, or diluent.

Another aspect of the disclosure provides a method of determining the susceptibility of a cell exhibiting a proliferative disorder to γ₁34.5 deficient herpes simplex virus-1 cytotoxicity comprising measuring the activity of the MEK signaling pathway in the cell, wherein an active MEK signaling pathway is indicative of the susceptibility of the cell to γ₁34.5 deficient HSV cytotoxicity. In some embodiments, the activity of the MEK signaling pathway in the cell is measured by determining the level of a phosphorylated form of a protein selected from the group consisting of MEK1, MEK2, ERK 1, and ERK 2, and preferably selected from either MEK1 or MEK2. Some embodiments of this aspect of the disclosure involve the above-described method wherein the phosphorylated form of the protein is measured using an antibody specifically recognizing the phosphorylated form of the protein. The method described above may also involve measuring the activity of MEK signaling by determining the MEK haplotype, or partial genotype, of the cell, wherein a non-deficient MEK haplotype is indicative of an active MEK signaling pathway. In certain embodiments, the non-deficient MEK haplotype is homozygous wild-type MEK. Also in some embodiments, the method may involve a cell exhibiting a proliferative disorder that is a cancer cell. Also, the method described above may involve a γ₁34.5 deficient HSV that is an HSV lacking the capacity to express a full-length ICP34.5 at about a wild-type level of expression.

Another aspect of the disclosure provides a method of identifying a patient with a cell proliferative disorder that is amenable to treatment with a γ₁34.5 deficient HSV comprising obtaining a cell sample from the patient; and measuring the activity of the MEK signaling pathway in the cell, wherein an active MEK signaling pathway is indicative of a patient with a cell proliferative disorder that is amenable to treatment with a γ₁34.5 deficient HSV. In some embodiments, the activity being measured is the level of a phosphorylated form of a protein selected from the group consisting of MEK1, MEK2, ERK1 and ERK2, preferably MEK1 or MEK2. In some embodiments of this aspect of the disclosure the activity of the MEK signaling pathway is measured by determining the MEK genotype of the cell, wherein a non-deficient MEK genotype is indicative of an active MEK signaling pathway. In some embodiments, the γ₁34.5 deficient HSV is an HSV lacking the capacity to express a full-length ICP34.5 at about a wild-type level of expression. This aspect of the disclosure comprehends embodiments in which the cell proliferative disorder is a cancer, a rheumatoid arthritis or a macular degeneration, and preferably a cancer such as a solid tumor cancer or a metastasized cancer.

Yet another aspect of the disclosure is a use of a γ₁34.5 deficient HSV in the preparation of a medicament for the treatment of a patient with a cell proliferative disorder comprising combining the γ₁34.5 deficient HSV with a pharmaceutically acceptable adjuvant, carrier, or diluent.

Yet another aspect of the disclosure is a method of treating an MEK⁺ cell exhibiting a proliferative disorder comprising contacting the cell with a therapeutically effective amount of a γ₁34.5 deficient HSV. In some embodiments of this aspect of the disclosure, the activity being measured is the level of a phosphorylated form of a protein selected from the group consisting of MEK1, MEK2, ERK 1 and ERK 2, preferably MEK1 or MEK2. Some embodiments of this aspect involve practice of the above-described method wherein the activity of the MEK signaling pathway is measured by determining the MEK haplotype of the cell, wherein a non-deficient MEK haplotype is indicative of an active MEK signaling pathway. In some embodiments of the method, the γ₁34.5 deficient HSV is an HSV lacking the capacity to express a full-length ICP34.5 at about a wild-type level of expression. In some embodiments, the cell proliferative disorder is a cancer.

In yet another aspect, the disclosure provides a use of a γ₁34.5 deficient HSV in the preparation of a medicament for the treatment of a cell exhibiting a proliferative disorder comprising combining the γ₁34.5 deficient HSV with a pharmaceutically acceptable adjuvant, carrier, diluent or excipient. Pharmaceutically acceptable adjuvants, carrier, diluents, and excipients are known in the art.

Other features and advantages of the disclosure will be better understood by reference to the brief description of the drawing and the detailed description of the subject matter of the disclosure that follow.

### BRIEF DESCRIPTION OF THE DRAWING

FIG. 1. HSV R3616 viral yields in a variety of cells characteristic of a variety of tumors. Cells were exposed to 1 PFU/cell of R3616 in serum free medium for 2 hours, after which medium containing virus was removed and fresh medium containing 1% calf serum was added. At 36 hours post-infection, R3616 viral recovery was determined by standard plaque assay.

FIG. 2. Differential protein synthesis and activation of protein kinase R

(PKR) in R3616 infected cancer cell lines inversely correlates with constitutive MEK activation in uninfected cancer cell lines A. Cell lines were infected with 10 PFU/cell of HSV R3616. At 11 hours post-infection, the cells were rinsed, starved of methionine for one hour, and then incubated in methionine-free medium supplemented with 100 μCi of [³⁵ methionine per ml for two additional hours. At 14 hours post-infection, 20 μg of equilibrated protein lysates were electrophoretically separated in denaturing polyacrylamide gels, transferred to a PVDF membrane, and exposed to autoradiography film. B Cells were infected with 10 PFU/cell of R3616 and whole-cell lysates, harvested at 12 hours post-infection, were resolved by SDS-PAGE and immunoblotted with an antibody that recognizes the autophosphorylated form of PKR on Threonine 446. In the lower panel, after overnight serum starvation, uninfected total whole-cell lysates were resolved by SDS-PAGE and immunoblotted with an antibody against the total and phosphorylated forms of ERK on threonine 202 and tyrosine 204.

FIG. 3. Deletion of mutant N-ras in human fibrosarcoma cells restricts viral replication Replicate cultures of HT1080 and MCH603 cells were infected with 1 PFU of R3616 or HSV-1(F) viruses per cell in serum free medium for 2 hours, after which medium containing virus was removed and replaced with fresh medium containing 1% calf serum. At 36 hours post-infection, viral recovery was determined by standard plaque assay.

FIG. 4. Diminished [³⁵S]-methionine metabolic labeling in virus infected human fibrosarcoma cells deleted for mutant N-ras. Replicate cultures of HT1080 and MCH603 cells were infected with 10 PFU of R3616 or HSV-1(F) viruses per cell. At 11 hours post-infection, the cells were rinsed, starved of methionine for one hour, and then incubated in methionine-free medium m supplemented with 100 μCi of [³⁵S] methionine per ml for two additional hours. At 14 hours post-infection, 20 μg of equilibrated protein lysates were electrophoretically separated in denaturing polyacrylamide gels, transferred to a PVDF membrane and exposed to autoradiography film.

FIG. 5. Increased PKR and eIF-2α phosphorylation in human fibrosarcoma cells deleted for mutant N-ras during R3616 infection. Replicate cultures of HT1080 and MCH603 cells were exposed to 10 PFU of R3616 or HSV-1(F) viruses per cell. Cells were harvested at 14 hours post-infection and processed as described in Example 1. The electrophoretically separated proteins were immunoblotted with antibodies recognizing the phosphorylated form of PKR on threonine 446 and the phosphorylated form of eIF-2α on serine 51, as well as for total PKR and eIF-2α.

FIG. 6. Inhibition of MEK by the addition of PD98059 increases PKR autophosphorylation and suppresses the accumulation of a γ2 viral protein (gC) in HT1080 cells infected with R3616. Replicate cultures of serum-starved HT1080 cells were infected with 10 PFU of R3616 viruses per cell in the presence or absence of 40 μM PD98059, as described in Example 1. Cells were harvested at 12 hours post-infection and the electrophoretically separated proteins were immunoblotted with antibodies recognizing either immediate-early [α (ICP27)], early [β(UL42)]or late [γ(gC)] viral proteins. The same lysates were immunoblotted to determine the total and phosphorylated forms of ERK1 and ERK2 (phosphorylated on threonine 202/tyrosine 204) and PKR (phosphorylated on threonine 446).

FIG. 7. Differences in cytopathic effects in virus infected caMEK (constitutively active MEK) and dnMEK (dominant negative MEK) stable cell lines. Replicate cultures of HT-caMEK and HT-dnMEK cells were infected with 10 PFU of mock, R3616 or HSV-1(F) viruses per cell. Photos were taken at 12 hours post-infection.

FIG. 8. The effect of dnMEK and caMEK over-expression on R3616 viral recovery and PKR function during R3616 infection. A. Replicate cultures of HT-dnMEK, HT1080, and HT-caMEK cells were exposed to one PFU of R3616 virus per cell in serum-free medium for 2 hours, after which medium containing virus was removed and fresh medium containing 1% calf serum was added. At 36 hours post-infection, R3616 viral recovery was determined by standard plaque assay B. To determine the influence of mutant MEK expression on PKR activation, replicate cultures of HT-dnMEK, HT1080 and HT-caMEK cells were exposed to 10 PFU of R3616 virus per cell. Cells were harvested at 12 hours post-infection and processed as described in Example 1. Electrophoretically separated proteins were immunoblotted with antibodies recognizing the total and phosphorylated form of the following proteins: ERK1 and ERK2 (phosphorylated on threonine 202 and tyrosine204), PKR (phosphorylated on threonine 446), and eIF-2α (phosphorylated on serine 51). The same lysates were immunoblotted with antibodies recognizing immediate-early [α (ICP27)] and late [γ(gC)] viral proteins. C. R3616 viral recovery from replicate cultures of Mia-dnMEK, MiaPaCa2 and Mia-caMEK at 36 hours post-infection. D. Immunoblotting was performed on replicate lysates of the Mia-dnMEK, MiaPaCa2 and MiacaMEK cells described in Section B, above.

FIG. 9. Diminished [³⁵S]-methionine metabolic labeling in R3616 infected human fibrosarcoma cells expressing dnMEK. Replicate cultures of HT-caMEK and HTdnMEKcells were infected with 10 PFU of R3616 or HSV-1(F) viruses per cell. At 11 hours post-infection, mock and virus infected cells were rinsed, starved of methionine for one hour, and then incubated in methionine-free medium supplemented with 100 [μCi of [³⁵S] methionine per ml for two additional hours. At 14 hours post-infection, 20 μg of equilibrated protein lysates were electrophoretically separated in denaturing polyacrylamide gels, transferred to a PVDF membrane and exposed to autoradiography film.

FIG. 10. Bioluminescence of systemically delivered R2636 in mice growing bilateral dnMEK- and caMEK-expressing tumor xenografts. HT-dnMEK and HT-caMEK tumors were established in the left and right hind limbs of athymic nude mice. Once tumors reached an average volume of 350 mm³ animals were given a single intraperitoneal injection of 9×10⁸ PFU of R2636 virus. Bioluminescence imaging was performed 5 days after intraperitoneal injection.

FIG. 11. A model for the interaction between activated MEK and the suppression of PKR function during viral infection of tumor cells by γ₁34.5 mutant viruses. Activation of the extracellular signal-regulated kinase (ERK)-kinase (MEK)/ERK pathway (i.e., the MAPK pathway) by either oncogenic activating mutations of Ras isoforms, point mutations within B-Raf alleles, or receptor tyrosine kinase activation/overexpression have been shown to be involved in transformation and tumor progression. In addition, Ras-independent activation of Raf/MEK/ERK signaling is cell- and tumor type-specific. This Figure schematically illustrates that activated MEK suppresses PKR auto-phosphorylation and effectively blocks PKR-mediated eIF-2a phosphorylation. Tumor cells with activated MEK/ERK signaling, therefore, are exquisitely permissive to A₁₁34.5 mutant viral replication and oncolysis.

FIG. 12. In tumor regrowth studies, systemic delivery of R3616 by intraperitoneal injection resulted in oncolysis of xenografts dependent on tumor MEK activity. Tumor xenografts were established in the hindlimbs of nude mice by injection of 5×10⁶ cells per animal. Tumor volume was determined by direct caliper measurement. Once tumors reached a mean volume of 115-150 mm³, animals were treated on day 0 and day 5 with 2×10⁶, 2×10⁷, or 2×10⁸ PFU intraperitoneal or 10⁸ PFU intratumoral R3616. Tumor growth was measured by calculating the ratio of tumor volume V to initial tumor volume V₀. A) HT-caMEK B) HT-dnMEK C) Hep3B (high MEK activity) D) PC-3 (low MEK activity)

FIG. 13. In vivo luciferase imaging of R2636 replication shows that HT-caMEK tumors permitted increasing viral replication and HT-dnMEK tumors restricted viral replication. Intraperitoneal administration of R2636 in HT-caMEK tumor bearing mice allowed viral localization to the hindlimb xenograft and subsequent replication. Tumor xenografts were established as described previously. Mice were injected with intratumoral (5×10⁷ PFU) or intraperitoneal (10⁸ PFU) R2636. On days 1, 3, 8, 12, and 22 following R2636 treatment, imaging of luciferase activity was performed on a charge-coupled device camera 15 minutes following IP injection of D-luciferin at 15 mg/kg body weight. A) HT-caMEK, intratumoral B) HT-dnMEK, intratumoral C) HT-caMEK, intraperitoneal D) HT-dnMEK, intraperitoneal.

FIG. 14. Quantified luciferase activity from HT-caMEK and HT-dnMEK tumor-bearing mice treated with 5×10⁷ PFU intratumoral or 10⁸ PFU intraperitoneal R2636. Using image analysis software to process images generated from R2636-treated mice bearing HT-caMEK and HT-dnMEK xenografts, luminescence was quantified as total photon flux, calculated using an area-under-the-curve analysis (MetaMorph). The baseline luminescence in the untreated HT-caMEK tumors was 1.8×10⁵±5.9×10³ photons. In HT-caMEK tumors injected intratumorally with 5×10⁷ PFU of R2636, the measured photon activity was 1.8×10⁶±6.6×10⁵, 1.1×10⁷±3.9×10⁶, 2.7×1.0⁶±1.2×10⁶, 4.3×10⁶±3.1×10⁶, and 1.6×10⁷±6.7×10⁶ on days 1, 3, 8, 12, and 22 respectively (p=0.042, 0.0208, 0.0726, 0.2149, and 0.0477, respectively, with reference to baseline luminescence in untreated control mice bearing HT-caMEK tumors). HT-caMEK xenografts treated with 10⁸ PFU of intraperitoneal R2636 resulted in measured photon emission of 6.6×10⁵±1.1×10⁵, 2.4×10⁶±1.1×10⁶, 8.4×10⁶±2.7×10⁶, 1.1×10⁷±5.0×10⁶, and 4.8×10⁷±2.1×10⁷ on days 1, 3, 8, 12, and 22, respectively (p=0.0019, 0.064, 0.0163, 0.0557, and 0.0499, respectively, with reference to untreated control tumor-bearing mice). In untreated control mice bearing HT-dnMEK tumors, baseline luminescence was 9.9×10⁴±1.3×10⁴ photons. HT-dnMEK xenografts injected intratumorally with 5×10⁷ PFU R2636 resulted in measured photon activity of 4.0×10⁶±1.6×10⁶, 6.8×10⁵±2.3×10⁵, 6.9×10⁵±5.0×10⁵, 9.4×10⁵±7.9×10⁵, and 3.2×10⁶±2.8×10⁶ on days 1, 3, 8, 12, and 22, respectively. HT-dnMEK xenografts treated with 10⁸ PFU intraperitoneal R2636 resulted in measured photon activity of 5.0×10⁵±1.4×10⁵, 2.6×10⁵±7.3×10⁴, 2.0×10⁵±1.5×10⁵, 4.2×10⁴±4.1×10³, and 4.4×10⁴±1.9×10³ on days 1, 3, 8, 12, and 22, respectively.

FIG. 15. Immunohistochemistry of HT-caMEK tumor for HSV-1 antigen 5 days following R3616 treatment demonstrated a different pattern of viral spread with intratumoral versus intraperitoneal injection. HT-caMEK xenografts were harvested 5 days following intratumoral (5×10⁷ PFU) or intraperitoneal (10⁸ PFU) injection of R3616. Tumors were formalin-fixed, paraffin-embedded, and probed with anti-HSV-1 antibody. A) Intratumoral injection (low and high power) showed viral spread outward from the needle track. B) Intraperitoneal injection showed a more diffuse pattern with multiple foci of replication.

FIG. 16. Viral recovery from HT-caMEK tumors 5 days following intratumoral injection with 5×10⁷ PFU R3616 or 10⁸ PFU R3616 was comparable. HT-caMEK xenografts were harvested 5 days post-treatment with either intratumoral 5×10⁷ PFU or intraperitoneal 10⁸ PFU of R3616. Viral titers from homogenized samples were determined by standard plaque formation assays on Vero cell monolayers.

FIG. 17. The effect of R2660 mutant virus on PC-3 tumors. Panel A: schematic representation of the R3616 and R2660 mutant viruses. Panel B: schematic representation of the test protocol. Panel C: measurements of the tumor volume after treatment.

### DETAILED DESCRIPTION

The present invention provides materials and methods for identifying target cells exhibiting a cell proliferation disease, disorder or condition that are amenable to herpes simplex virus-based therapy. The HSV useful in methods of the invention do not express wild-type levels of ICP34.5 and, for that reason, are relatively safe, as exhibited by the attenuated virulence of such HSV. In identifying those cells that not only exhibit a cell proliferative disease, disorder or condition, but also have an active MAPK pathway, e.g., are MEK⁺, the methods of the invention facilitate the identification or diagnosis of those diseases, disorder or conditions amenable to treatment with such HSV. Methods of treating such diseases, disorders or conditions, as well as methods of ameliorating a symptom of such a disease, disorder or condition and methods of preventing such diseases, disorders or conditions, are other beneficial aspects of the invention. In combining HSVs having cytotoxic effects that are relatively specific to cells exhibiting cell proliferative disorders with target cells having an active MAPK pathway, e.g., Ras/Raf/MEK/ERK pathway, the invention provides methods for identifying or diagnosing cell diseases, disorders or conditions best suited to treatment with the modified HSV, as well as methods of preventing, treating, or ameliorating at least one symptom associated with such disease, disorder or condition.

Studies described herein demonstrate that transduction of a cell line with a constitutively active mitogen-activated protein kinase (MAPK) kinase (MEK) coding region conferred susceptibility to a γ₁34.5 deficient HSV, such as the HSV R3616 virus, whereas cells transduced with a dominant negative MEK coding region became more resistant to the recombinant virus (Smith et al., J. Virol. 80:1110-1120 (2006)). MEK is a key regulator in the MAPK pathway and is activated by MAPK kinase kinases (A-RAF, B-RAF, and RAF-1) which are downstream of RAS. MEK, in turn, phosphorylates its only known substrates, the MAPKs (ERK1 and ERK2). MEK is constitutively activated in a wide variety of tumors, and functions to promote cell survival (Ballif et al., Cell Growth Differ. 12:397-408 (2001), Von Gise et al., Mol. Cell. Biol. 21:2324-2336 (2001), and Xia et al., Science 270:1326-1331 (2001)) and to protect tumor cells from multiple apoptotic stimuli. Extensive analyses of the phenotype of the parent and transduced tumor cells exposed to the γ₁34.5 mutant virus indicated that in cells transduced with the constitutively active MEK, PKR is not activated, in contrast to cells transduced with the dominant negative MEK. Further consideration of the disclosure of the invention will be facilitated by a consideration of the following express definitions of terms used herein.

An “abnormal condition” is broadly defined to include mammalian diseases, mammalian disorders and any abnormal state of mammalian health (i.e., a mammalian condition) that is characterized by abnormal cell proliferation in an animal, such as man, relative to a healthy individual of that species. Preferably, the abnormal cell proliferation involves excess cell proliferation. Exemplary conditions include any of the wide variety of cancers afflicting humans or other animal species (e.g., mammalian species), including solid tumors and metastasized cancers, as well as rheumatoid arthritis, macular degeneration, and the like.

“Administering” is given its ordinary and accustomed meaning of delivery by any suitable means recognized in the art. Exemplary forms of administering include oral delivery, anal delivery, direct puncture or injection, including intravenous, intraperitoneal, intramuscular, subcutaneous, intratumoral, and other forms of injection, spray (e.g., nebulizing spray), gel or fluid application to an eye, ear, nose, mouth, anus or urethral opening, and cannulation.

An “effective dose” is that amount of a substance that provides a beneficial effect on the organism receiving the dose and may vary depending upon the purpose of administering the dose, the size and condition of the organism receiving the dose, and other variables recognized in the art as relevant to a determination of an effective dose. The process of determining an effective dose involves routine optimization procedures that are within the skill in the art.

An “animal” is given its conventional meaning of a non-plant, non-protist living being. A preferred animal is a mammal, such as a human.

“Ameliorating” means reducing the degree or severity of, consistent with its ordinary and accustomed meaning.

“Pharmaceutical composition” means a formulation of compounds suitable for therapeutic administration, to a living animal, such as a human patient. Typical pharmaceutical compositions comprise a therapeutic agent such as an HSV virus not elaborating a wild-type level of active ICP34.5, in combination with an adjuvant, excipient, carrier, or diluent recognized in the art as compatible with delivery or administration to an animal, e.g., a human.

“Adjuvants,” “excipients,” “carriers,” and “diluents” are each given the meanings those terms have acquired in the art. An adjuvant is one or more substances that serve to prolong the immunogenicity of a co-administered immunogen. An excipient is an inert substance that serves as a vehicle, and/or diluent, for a therapeutic agent. A carrier is one or more substances that facilitates manipulation of a substance (e.g., a therapeutic), such as by translocation of a substance being carried. A diluent is one or more substances that reduce the concentration of, or dilute, a given substance exposed to the diluent.

“Media” and “medium” are used to refer to cell culture medium and to cell culture media throughout the application. As used herein, “media” and “medium” may be used interchangeably with respect to number, with the singular or plural number of the nouns becoming apparent upon consideration of the context of each usage.

Mindful of the preceding definitions, it is noted that herpes simplex virus mutants lacking the γ₁34.5 gene, or lacking the capacity to express active ICP34.5, are not destructive to normal tissues but are potent cytolytic agents in human tumor cells in which the activation of protein kinase R (PKR) is suppressed. Thus, replication of a Δ₁34.5 mutant (R3616) in 12 genetically defined cancer cell lines correlated with suppression of PKR but not with the haplotype of Ras (i.e., the Ras-specific genotype). Extensive analyses of two cell lines transduced with either dominant negative MEK (dnMEK) or constitutively active MEK (caMEK) indicated that in R3616 mutant infected cells, dnMEK enabled PKR activation and decreased virus yields, whereas caMEK suppressed PKR and enabled better viral replication and cell destruction in transduced cells in vitro or in mouse xenografts. The results indicated that activated MEK mediated the suppression of PKR and that the status of MEK predicts the ability of γ₁34.5 mutant viruses to replicate and destroy tumor cells. In addition, γ₁34.5 mutant HSV comprising one or more coding regions for the expression of heterologous gene product(s) are useful in effectively converting or ensuring that a tumor cell exhibits a suppressed PKR phenotype, thereby rendering such a cell susceptible to destruction by the γ₁34.5 mutant HSV.

PKR appears to play a key role in conferring resistance to γ₁34.5 mutants. The importance of PKR to a cell's innate antiviral response to viral infection is underscored by the observation that γ₁34.5 mutants replicate to near wild-type levels in murine embryonal fibroblast (MEF) cells derived from mice lacking PKR. Moreover, γ₁34.5 HSV mutants are virulent in PKR^{-/-} mice, but not in wild-type mice. In addition, exogenous a interferon (INF-α) effectively suppresses γ₁34.5 mutant replication in PKR^{+/+} MEFs, but has no effect in PKR^{-/-} MEFs, while wild-type HSV-1 was reported to be resistant to the anti-viral effects of IFN in these cells. Therefore, replication of mutants lacking γ₁34.5 is largely dependent on the ability of cells to activate PKR-dependent pathways of host cell defense.

PKR also exerts potent growth suppressive effects and apoptotic cell death effects induced by multiple stimuli. Alternatively, inhibition of PKR function by over-expression of catalytically inactive mutants of PKR and eIF-2α, transformed NIH 3T3 cells and primary human cells when co-expressed with large T antigen and human telomerase reverse transcriptase (hTERT) in a manner similar to the necessary mitogenic signal transmitted by activated Ras.

Growth factor withdrawal also induces PKR activation, eIF-2α phosphorylation and apoptosis in several growth factor-dependent hematopoietic cell lines. Growth factor withdrawal also downregulated the activity of MEK, a critical downstream Ras effector kinase, while overexpression of constitutively active MEK mutants protected growth factor-dependent cell lines from multiple apoptotic stimuli, including growth factor withdrawal. MEK is a key regulatory kinase activated by MAPK-kinase-kinases (A-Raf, B-Raf and Raf-1) that functions to promote cell survival. Accordingly, MEK and its only known substrate, MAPKs (ERK1 and ERK 2) are constitutively activated in a large percentage of tumors as a consequence of dysregulated growth factor secretion, tyrosine kinase receptor activation, activating mutations in Ras isoforms and somatic activating missense mutations of B-Raf.

The data disclosed herein establish that PKR activation is suppressed in a subset of cancer cells, thereby rendering them susceptible to viral replication and cytolysis by a γ₁34.5 mutant HSV, e.g., HSV R3616. Using pharmacologic inhibitors of MEK and catalytically active and inactive mutants of MEK, constitutive MEK activity was shown to suppress the viral activation of PKR. The status of MEK correlates with the ability of tumor cells to support the replication of γ₁34.5 mutant HSV viruses and that replication ultimately destroys the host tumor cells. Accordingly, the status of MEK is predictive of those cancer cells most susceptible to destruction by HSV viruses not elaborating wild-type levels of active ICP34.5.

The invention contemplates any herpes simplex virus, including HSV-1, HSV-2 and hybrids thereof, that does not express a wild-type level of ICP34.5, although it is preferred that the HSV is an HSV-1. Derivatives of these viruses are also contemplated by the invention, provided such derivatives both retain the capacity to exert a cytotoxic or cytopathic effect (i.e., lytically infect) in at least one tumor cell type and do not express a wild-type level of ICP34.5. Suitable viral derivatives include HSV having at least one mutation, silent or not, in addition to any mutation associated with the failure to express a wild-type level of ICP34.5, as well as viral fragments. Preferably, such viral derivatives retain the ability to form infectious virion, eliminating the need for engineered forms of delivering the viral agent.

The invention also comprehends HSV having any known mechanism of reducing the level of expressed, active ICP34.5 below wild-type levels including, but not limited to, γ₁34.5 deletion mutants (i.e., γ₁34.5 mutants) that either express a truncated gene product of reduced or undetectable activity or that do not express any gene product. Alternatively, or in conjunction with a deletion mutant, the invention contemplates an insertion mutant that reduces or eliminates the ICP34.5 activity of any expressed gene product, missense or nonsense mutations that eliminate or reduce expressed ICP34.5 activity in terms of either the level or stability of such activity, second-site mutations such as the insertion of an anti-sense coding region in the HSV genome, non-coding region mutations affecting the expression control of γ₁34.5 such as a down-regulating mutation in a promoter affecting γ₁34.5 expression, or any other HSV modification known in the art to reduce the level of expressed ICP34.5 activity below wild-type levels. Preferably, the modification of HSV, e.g., the mutation, is present in each copy of the relevant genetic element (e.g., a mutation in the coding region of γ₁34.5 is preferably found in both copies of γ₁34.5 found in the HSV genome). The invention also embraces singular modifications of HSV where the genetic element is naturally present as a single copy in HSV or where an HSV derivative has been rendered hemizygous for the relevant genetic element. Preferably, the level of expressed ICP34.5 is reduced below detectable levels.

With respect to heterologous coding regions, the invention contemplates a variety of coding regions useful in effectively suppressing PKR when expressed. Suitable heterologous coding regions include the coding region Dora functional member of the MAPK (Ras/Raf/ MEK/ERK) pathway, and preferably a constitutively active member of the pathway. Exemplary Ras coding regions encode any of wild-type N-Ras (SEQ ID NO:7 encoding SEQ ID NO:8), K-Ras (SEQ ID NO:9 encoding SEQ ID NO:10) or H-Ras (SEQ ID NO:11 encoding SEQ ID NO:12), as well as mutant active Ras isoform variants. For compact yet complete disclosure, wild-type sequences of members of the MAPK pathway are provided and the sequence differences from wild-type are indicated for the variants. The most common mutations are at residues C/G12, G13 and Q61. There are numerous examples of active mutant Ras isoforms known in the art including, but not limited to, K-RasV12, K-RasD12, K-RasG12, H-RasV12, K-RasD13, and N-RasV12 (Bos, 49(17):4682-9, 1989, incorporated herein by reference).

Exemplary Raf coding regions encode any one of the wild-type forms of Raf (SEQ ID NO:13 encoding SEQ ID NO:14 for B-Raf), Raf-CAAX (Leevers et al., Nature 369(6479):411-4, 1994, incorporated herein by reference), RafS338A (Diaz et al., Molecular and Cellular Biology 17(8):4509, 1997; incorporated herein by reference), RafS339A (Diaz et al., (1997); incorporated herein by reference), or Raf BXB (Bruder et a)., Genes & Dev. 6:545-556, 1992, incorporated herein by reference. Further, the invention embraces V600E B-Raf (Andersen et al., Cancer Res. 1 64:5456-60, 2004, incorporated herein by reference notwithstanding the identification therein to V599E due to a sequence error in the publication). The variations from the wild-type Raf sequence found in any of Raf-CAAX, RafS338A, RafS339A , Raf BXB, and V600E B-Raf can be present in any combination. Two isoforms of MEK are found in humans, i.e., MEK1 and MEK2. The invention comprehends wild-type MEK1 (SEQ ID NO:1, encoding SEQ ID NO:2) and wild-type MEK2 (SEQ ID NO:5 encoding SEQ ID NO:6). Also contemplated are active mutant MEKs, including constitutively active MEKs. Examples of active mutants known in the art and embraced by the invention include ΔN3MKK1 S218E/S222D, an N-terminal truncation mutant of MEK1 that also includes missense mutations at residues 218 and 222; an analogous variant (N-terminal truncation and amino acid substitutions at the equivalent of positions 218 and 222 of MEK1) of MEK2 is also contemplated (Mansour, et al., Science 265(5174):966-70, 1994, incorporated herein by reference). Further, full-length MEK1 and MEK2 proteins containing a missense mutation yielding S281E or S222D, and preferably both mutations, are contemplated.

The ERK component of the MAPK pathway is present in two isoforms, ERK1 and ERK2, in humans. Contemplated by the invention are HSV comprising coding regions for wild-type ERK, including wild-type human ERK1 (SEQ ID NOS:15 and 17 encode SEQ ID NOS:16 and 18, respectively, with SEQ ID NOS:15 and 16 relating to transcript variant 1 and SEQ ID NOS:17 and 18 relating to transcript variant 2) and/or ERK2 (SEQ ID NO:3 encodes SEQ ID NO:4 of ERK2) (Emrick, et al., J. Biol. Chem. 276:46469-46479, 2001, incorporated herein by reference). Exemplary variants of ERK2 include, but are not limited to, variants known in the art such as variants containing an amino acid substitution at E58Q, D122A, S151A, or S221A (Zhang, et al., J. Biol. Chem. 278: 29901-29912, 2003, incorporated herein by reference), as well as S151D or L73P (Emrick et al., supra).

In addition to the foregoing wild-type and variant members of the MAPK pathway, the HSV according to the invention may comprise fusion proteins, such as a MEK2-ERK1 fusion as described in Robinson, et al., Curr. Biol. 8:1141-1150, 1998, incorporated herein by reference. The MEK2-ERK1 fusion of Robinson et al. encodes a full length MEK2 (SEQ ID NO:6 encoded, e.g., by SEQ ID NO:5) fused to a coding region for a linker, such as a ten-amino acid linker (Glu-Gly), in turn fused to a full-length ERK1 (SE( )ID NO:16 or 18 encoded, e.g., by SEQ ID NO:15 or 17, respectively). The linker can vary in length and/or sequence, provided that it is compatible with secondary and tertiary structure formation required for activity as an ultimate suppressor of PKR activity. Also contemplated are full-length fusions of MEK1-ERK1, MEK2-ERK2, MEK1-ERK2 and fusions in which the orientation of the two proteins are reversed, along with a linker conforming to the requirements provided above. Collectively, each of the MEK1/2-ERK1/2 and ERK1/2-MEK1/2 fusions is referred to herein as a MEK-ERK fusion. Further, N-terminally deleted MEK1 or MEK2, particularly N-terminal deletions of the four leucine residues contributing to the nuclear export signal, as described in Robinson et al., supra, incorporated herein by reference, are contemplated as elements of MEK-ERK fusions. In addition, conservative coding regions specifying amino acids that are conservative substitutions for the above-identified wild-type variants are envisaged (e.g., any conservative substitution for the serine residues as positions 218 and 222 in the above-described upregulated MEK variants is contemplated). In the present context, a conservative substitution preferably conforms to conventional understanding and more preferably conserves the functional characteristic (contribution to activity level) of the amino acid being substituted, such as the like susceptibility to phosphorylation of S, T, Y and other phosphorylatable amino acids (D, E, H). Non-conservative substitutions, deletions and insertions (relative to wild-type counterparts rather than the upregulated variants described above) that result in upregulated activity of the MAPK pathway are also comprehended, such as those non-conservative substitutions, deletions and insertions of coding regions of the MAPK pathway known in the art.

Beyond the various coding regions of the MAPK pathway, HSVs according to the invention may comprise a heterologous (foreign to wild-type HSV) coding region for a catalytically inactive mutant of PKR or for a catalytically inactive mutant of eIF-2α, as known in the art. Further, HSV comprising a coding region for a growth factor, the overexpression of which is known in the art to result in upregulated activity of the MAPK pathway is suitable, as is an active mutant of a tyrosine kinase receptor that is known in the art to regulate the activity of the MAPK pathway.

The methods of the invention comprehend any process or assay known in the art for detecting or measuring a protein indicative of the status of a MAPK pathway in a cell. Suitable proteins include, but are not limited to, members of the Ras/Raf/MEK/ERK module of the MAPK pathway, e.g., any form of Ras, a G-protein specifically interacting with any such form of Ras, Raf (A-Raf, B-Raf and Raf-1; also referred to as Raf-A, Raf-B, and Raf-C, respectively), MEK1 (MKK1), MEK2 (MKK2), ERK1, and ERK2. Any known isoform of a protein involved in a MAPK pathway may be the sole component detected or measured, or may be one of a plurality of elements detected or measured, for example in the context of assays measuring a plurality of isoforms of a given protein or assays collectively measuring one or more isoforms of at least two proteins in a MAPK pathway. In preferred embodiments, the proteins being detected or measured are phosphorylated derivatives of the proteins, wherein the phosphorylation is known in the art to be associated with activation of that protein. Further, it is expected that accessory proteins in a MAPK pathway, e.g., exchange factors, modulators, scaffolding molecules, adapter proteins, and/or chaperones, that are known to vary in activity (whether that variance is attributable to changes in specific activity or active protein level) in a manner predictive of MAPK pathway activation, may also serve alone or in combination with other suitable proteins as the basis for detecting and/or measuring MAPK pathway status. Exemplary accessory proteins include, but are not limited to, MEKK-1, mos, Tp1-2, SOS, SUR-8, KSR, PBS2, 14-3-3, Hsp90, Hsp50/Cdc37, FKBP65, Bag-1, Rsk-1, and proteins identified in Kolch, W., Nat. Rev. Cell Biol. 6:827-837 (2005), incorporated herein by reference. Preferred accessory proteins are human proteins identified above and human orthologs of non-human proteins identified above. In other processes of the invention, comparative measures of one or more isoforms of one or more MAPK pathway proteins is obtained to provide a comparative measure indicative of MAPK pathway status. Preferred proteins for use in any of these processes include MEK1, MEK2, ERK1 and ERK2.

Yet other processes according to the invention involve haplotyping a target cell, by which is meant the partial or complete characterization of at least one genetic element involved in the expression of at least one isoform of a MAPK pathway protein indicative of MAPK pathway status. The characterizations will typically provide partial or complete sequence information for at least one genetic element, which may be obtained by any method known in the art, including but not limited to chemical or enzymatic sequencing techniques, whether automated or not. Also contemplated are hybridization-based technologies using one or more probes of any suitable length and under any suitable hybridization conditions that are compatible with the reliable identification of a particular genetic element predictive, alone or in combination with additional information, of MAPK pathway status. Preferably, the probe is an oligonucleotide of 8-50 nucleotides and stringent hybridization conditions are employed to facilitate the inferential determination of at least a partial sequence diagnostic of MAPK pathway status. Also included in the haplotyping processes of the invention are genetic complementation studies in which distinct naturally existing, or engineered, phenotypes are associated with the relevant haplotypes. Any other process known in the art for determining the absolute or relative level of activity of at least one isoform of a protein in a MAPK pathway that is predictive of MAPK pathway status is also embraced by the invention.

The invention also provides methods of treating diseases, disorders or conditions characterized by abnormal cell proliferation, typically hyperproliferation, provided that the abnormally proliferating cells have a MAPK pathway of active status. Diseases, disorders or conditions suitable for treatment include any form of cancer, including solid-tumor cancers such as inoperably located tumors or metastasized cancers, as well as rheumatoid arthritis, macular degeneration, and any disease, disorder or condition characterized by abnormal cell proliferation, as would be understood in the art, provided the cells have an active MAPK pathway. A related aspect of the invention provides methods for ameliorating at least one symptom associated with such disease, disorder or condition. For example, the invention contemplates administering an effective dose of an HSV that does not express a wild-type level of active ICP34.5 to an organism suffering from a cancerous condition due to MAPK-active cancer cells, wherein the dose is sufficient to reduce the pain, swelling, or other physiological symptom attending tumor growth. A benefit provided by these methods of the invention is that the HSV therapeutic is effective in embodiments of the disease, disorder or condition that have proven refractory to treatment with conventional therapies, such as inoperable tumors of the brain or other inaccessible regions of a body as well as metastasized cancers.

The invention further contemplates prophylactic methods wherein a dose of an HSV, as described above, that is known to be effective in ameliorating a symptom or treating a disease, disorder or condition characterized by abnormal ccll proliferation is administered to an organism at risk of developing such a disease, disorder or condition.

Administration of the above-described HSV compositions according to the invention is by any known route, provided that the target cell or tissue is accessible via that route. Notably, the experimental results disclosed herein establish that two isogenic tumor cell lines differing in susceptibility to the γ₁34.5 mutant R3616 were used to study the distribution and persistence of virus delivered by different routes. As expected, the virus replicated better and persisted longer in the susceptible (high MEK activity) tumors in mouse xenografts. A significant finding was that systemic administration to the tumor-bearing mouse was as effective as intratumoral delivery with regard to tumor oncolysis. Accordingly, the pharmaceutical compositions may be introduced into the subject by any conventional method, e.g., by intravenous, intradermal, intramuscular, intramammary, intraperitoneal, intrathecal, retrobulbar, intravesicular, intrapulmonary (e.g., term release); sublingual, nasal, anal, vaginal, or transdermal delivery, or by surgical implantation at a particular site. The treatment may consist of a single dose or a plurality of doses over a period of time.

Upon formulation, solutions are administered in a manner compatible with the dosage formulation and in such amount as is therapeutically effective. Appropriate dosages may be ascertained through the use of established routine assays. As studies are conducted, further information will emerge regarding optimal dosage levels and duration of treatment for specific diseases, disorders, and conditions.

In preferred embodiments, the unit dose may be calculated in terms of the dose of viral particles being administered. Viral doses are defined as a particular number of virus particles or plaque forming units (pfu). Particular unit doses include 10³, 10⁴, 10⁵, 10⁶, 10⁷, 10⁸, 10⁹, 10¹⁰, 10¹¹, 10¹²v, 10¹³ or 10¹⁴ pfu. Particle doses may be somewhat higher (10- to 100-fold) due to the presence of infection-defective particles, which is determinable by routine assays known in the art.

The pharmaceutical compositions and methods of the invention are useful in the fields of human medicine and veterinary medicine. Thus, the subject to be treated (whether to treat or prevent a disease, disorder or condition, or to ameliorate a symptom thereof) may be a vertebrate, e.g., a mammal, preferably human. For veterinary purposes, subjects include, for example, farm animals such as cows, sheep, pigs, horses and goats, companion animals such as dogs and cats, exotic and/or zoo animals, laboratory animals including mice, rats, rabbits, guinea pigs and hamsters; and poultry such as chickens, turkey, ducks and geese.

Having provided a general description of the various aspects of the invention, the following disclosure provides examples illustrative of the invention, wherein Example 1 describes the materials and methods used in conducting the studies reported herein, Example 2 discloses data establishing the correlation of γ₁34.5 deficient HSV replication and the MAPK (e.g., MEK) phenotype of host cells, Example 3 reveals that an N-Ras mutation enables efficient replication of R3616 mutant HSV virus in human fibrosarcoma cells; Example 4 discloses that the inhibition of MEK by PD98059 (a known MEK inhibitor) resulted in increased levels of PKR phosphorylation, decreased viral protein accumulation, and diminished replication of mutant HSV virus R3616; Example 5 discloses data showing that viral activation of PKR by mutant HSV R3616 is suppressed in tumor cell lines that overexpressed constitutively active MEK, while expression of dominant negative MEK increased PKR activation and restricted R3616 viral replication; Example 6 establishes that intratumoral inoculation of R3616 mutant HSV virus resulted in tumor regression in tumors expressing caMEK, but not in tumors expressing dnMEK; Example 7 shows that the systemic administration of a recombinant HSV virus R2636, expressing the gC-Luc construct, targeted tumor tissue overexpressing constitutively active ME}{; and Example 8 reveals that various routes of administration of mutant HSV, including systemic delivery, are suitable for the treatment of MEK-overexpressing tumors.

### EXAMPLE 1

### Materials and Methods

Molecular Constructs—Constitutively active MEK-1-encoding (caMEK) and dominant negative MEK-1-encoding (dnMEK) plasmids, designated pNC84 and pNC92, respectively, were provided by J. Charron (Quebec, Canada). Their constructions are detailed in Ref. 34, incorporated herein by reference. Briefly, coding sequences for serine residues 218 and 222 of human wild-type MEK-1 were mutated either to aspartic acid residues (D218S and D222S), creating a constitutively active, phosphomimetic mutant, or to alanine residues (A218S and A222S) to create a dominant negative-functioning kinase mutant. The mutant MEK-1 cDNAs contain an in-frame FLAG epitope at the N-terminus under the transcriptional control of a CMV promoter in the pCMV-Tag2b mammalian expression vector (Qiagen Inc. Valencia, Calif.). Orientation and cDNA insert sequence were confirmed by DNA sequencing.

Cell Culture—PC-3 and DU145 (human prostate cancer), Panc-1, BxPc3, and MiaPaCa2 (human pancreatic cancer) MCF7 and MDA-MB-231 (human breast cancer), DLD-1 and WiDr (Colorectal cancer), Hep3B (human hepatoma), Vero (Green Monkey Kidney) cell lines were originally obtained from the American Type Culture Collection (Manassas, Va.). The Huh7 hepatoma cell line was originally obtained from J. R. Wands (Harvard Medical School, Boston, Mass., USA). The HT1080 (human fibrosarcoma) cell line containing one wild-type and one oncogenic (Q61K) N-ras allele (1, 40) was also obtained from the American Type Culture Collection. HT1080 cells having lost the activated mutant N-ras allele were obtained from E J, Stanbridge (Irvine, Calif.) and have been described previously and published as MCH603 (40). HT-caMEK and HT-dnMEK are clonal cell lines constructed from the parental cell line HT 1080, a human fibrosarcoma. The methods of transfection with genetic constructs pNC84 and pNC92, which express constitutively active and dominant negative MEK respectively, are described in Smith et al., J. Virol. 80:1110-1120 (2006) and Mansour et al., Biochem. 35:15529-15536 (1996), both incorporated herein by reference. The above cell lines were grown in DMEM (GIBCO/Invitrogen Corporation, Grand Island, N.Y.)/10%FCS (Intergen, Purchase, N.Y.)/1% penicillin-streptomycin at 37° C. and 7% CO₂. HT-caMEK and HT-dnMEK were grown in medium supplemented with 500 μg/ml of G418 (Geneticin, Gibco BRL).

For the experiments described in Example 9, PC-3 (human prostate cancer) cells were originally obtained from the American Type Culture Collection (Manassas, Va.) and grown in Dulbecco's Modified Eagle's Medium: Nutrient Mixture F-12 (Life Technologies/Invitrogen Corp., Grand Island, N.Y.) supplemented with 10% fetal calf serum (Intergen, Purchase, N.Y.) and 1% penicillin-streptomycin at 37° C. and 7% CO₂.

Viruses—HSV-1(F) is the prototype wild-type HSV-1 strain (18). The derivation and properties of the recombinant virus R3616, which lacks both copies of the γ₁34.5 gene (11), and recombinant R2636 carrying the luciferase gene driven by the glycoprotein C (gC) promoter (gC-luc) in place of the γ₁34.5 gene, were reported in Nakamura et al. (ref. 38), and that description is incorporated herein by reference.

Construction of recombinant viruses (e.g., R2660, which carries caMEK driven by the glycoprotein C promoter together with the luciferase gene driven by the immediate-early CMV promoter (gC-caMEK/pCMV-Luc) in place of the γ₁34.5 genes). The recombinant virus R2660 was constructed in two steps. The first step involved the replacement of the thymidine kinase (TK) genes previously inserted in place of both copies of the γ₁34.5 gene in the recombinant virus R3659 with the pgC-caMEK/pCMV-Luc construct. The pgC is the inducible promoter for the gC gene of HSV, caMEK is a constitutively active MEK allele, pCMV is the immediate-early Cytomegalovirus promoter, and Luc is the gene encoding the enzyme luciferase, a marker. The procedure has been described by Post et al., Cell 25:227-232 (1981). The transfer plasmid was constructed as follows. Plasmid gC-pGL3 (Mezhir et al., Cancer Res. 65:9479-9484 (2005)) contained an HSV-1 glycoprotein C promoter inserted in the SacI-NheI sites upstream of the luciferase gene in the pGL3 vector (Promega). The immediate-early CMV promoter (pCMV) was excised from pRB5850 (Sciortino et al., Proc. Natl. Acad. Sci. (USA) 99:8318-8323 (2002)) and inserted into the XhoI-HindIII sites of gC-pGL3, placing it upstream of the luciferase gene, giving rise to pRB6031 (gC/pCMV-Luc). The flag-tagged caMEK sequence was derived from plasmid pNC84 as described in Smith et al., J. Virol. 80:1110-1120 (2006). pNC84 was digested with HindIII and KpnI restriction endonucleases, blunt-ended using Klenow enzyme (Klenow fragment of DNA polymerase, as known in the art), and religated. This step removed the KpnI and SalI sites from pNC84 to facilitate subsequent cloning. The resultant plasmid was digested with NotI and MluI restriction endonucleases and blunt-ended using Klenow enzyme. The DNA fragment containing flag-tagged caMEK sequence was purified using a Qiagen gel extraction kit and cloned into the NheI site of pRB6031, placing it downstream of the gC promoter. This plasmid (gC-caMEK/pCMV-Luc), designated pRB6033, was digested with KpnI and SalI restriction endonucleases. The DNA fragment containing the gC-caMEK/pCMV-Luc construct was purified using a Qiagen gel extraction kit, blunt-ended using the Klenow enzyme, and subcloned into the BstEII site of plasmid pRB3616. pRB3616 contains the HSV-1 BamHI S fragment with a deletion extending from the BstEII to the StuI site in the γ₁34.5 gene (Chou et al., Virol. 68:8301-8311 (1994)). The resultant plasmid was designated pRB6035. Recombinant virus R2653 was constructed as follows. Rabbit skin cells were transfected with R3659 viral DNA together with transfer plasmid pRB6035 containing the gC-caMEK/pCMV-Luc construct using Lipofectamine reagent (Life Technologies). Cells were harvested when they showed 100% cytopathic effect. The progeny of transfection were plated on 143 TK⁻ cells in the presence of bromodeoxyuridine to select for TK⁻ viruses. Viruses replicating under these conditions were screened by PCR, as described herein. Positive candidates were purified through four successive cycles of single plaque purification, verified to express MEK and luciferase, and then amplified.

In the next step, the TK gene was restored at its initial locus. Rabbit skin cells were cotransfected with R2653 (TK⁻ gC-caMEK/pCMV-Luc) viral DNA together with plasmid pRB103 carrying an HSV-1 TK gene in the BamHI Q fragment (Post et al., Proc. Natl. Acad. Sci. (USA) 77:4201-4205 (1980)). The progeny of transfection were plated on 143 TK⁻ cells in the presence of hypoxanthine/aminopterin/thymidine media to select for TK⁺ viruses. Viruses replicating under these conditions were first screened by PCR, as described herein. Viruses repaired to contain the TK gene were purified through four successive cycles of single plaque purification, and then amplified. Viral stock R2660 (TK⁺ gC-caMEK/pCMV-Luc) was prepared and titered on Vero cells.

Screening for recombinants by PCR. DNAs from virus plaques grown on Vero cells were subjected to PCR analyses (40 cycles of 95° C., 1 minute, 55° C. for 1 minute, 68° C. for 4 minutes) using pfu polymerase in 50 μl reaction mixtures with the following primers for initial screening of the MEK viruses. Primers A(gagtggttacgcgcggcgcg; SEQ ID NO:19) and MEK (gcagagctggtcccgttaactg; SEQ ID NO:20) amplified 1213-bp fragment while primers B (gcactactcgcctctgcacg; SEQ ID NO:21) and Luc2 (cgctgaattggaatccatcttg; SEQ ID NO:22) amplified 700-bp fragment from gC-MEK/CMV-Luc viruses. Primers TK1 (ccgcgtttatgaacaaacg; SEQ ID NO:23) and TK2 (gcagatcttggtggcgtg; SEQ ID NO:24) were used (35 cycles of 95° C., 1 minute/60° C., 1 minute/72° C., 3 minutes) for screening of viruses exhibiting a repaired TK (i.e., TK⁺ at the TK locus) gene.

Construction of stable cell lines—Mutant FLAG-tagged caMEK-1- or dnMEK-containing plasmids were transfected into replicate cultures of HT1080 or MiaPaCa2 cells on 60 mm dishes using Superfect Reagent (Qiagen Inc. Valencia, Calif.). Briefly, 5 μg of plasmid DNA was diluted in 300 μl of serum and antibiotic free DMEM, complexed with Superfect (20 μl) reagent for 10 minutes at room temperature and added to cells at 37° C. for 6 hours, after which medium was removed and replaced with DMEM containing 10% calf serum. After 24 hours of incubation, the cells were harvested, suspended in 5 ml of DMEM medium containing 10% FCS and 1 ml of this cellular suspension was grown on separate 100 mm dishes in a total volume of 10 ml of DMEM containing 10% calf serum supplemented with antibiotics (e.g., penicillin and streptomycin, each at conventional concentrations well-known in the art) and 800 μg/ml of G418 (Geneticin [Gibco BRL]). Medium containing G418 was replaced every four days until approximately 2 weeks after culture initiation, when cell colonies were visible and could be selected for clonal expansion using sterile cloning cylinders, as described in Gupta et al. (ref 22), which is incorporated herein by reference. The level of FLAG-MEK expression was assessed by immunoblotting 20 μg of equilibrated lysates from isolated clones using a monoclonal antibody to the FLAG epitope (Sigma Chemical Co,. St. Louis, Mo.). Clonal transfectants derived from the HT1080 parent cell line, designated HT-caMEK and HT-dnMEK, and from the MiaPaCa2 parent cell line, designated Mia-caMEK and Mia-dnMEK, with equivalent levels of FLAG-MEK expression, were chosen for further analysis.

Viral Infection—Cells were seeded onto 60 mm dishes at 1×10⁶ cells per dish. The next day cells were generally exposed to the viruses (1 or 10 plaque forming units per cell (PFU/cell)) for 2 hours at 37° C. and then removed and replaced with medium containing 1% calf serum. The infection continued at 37° C. for the length of time indicated for each experiment. Cells were either labeled for de 110V0 protein synthesis, harvested for immunoblotting, or collected for assaying viral recovery on Vero cell monolayers as previously described in Chou et al. (ref 11), incorporated herein by reference.

[³⁵S] Methionine Labeling—For metabolic labeling experiments, at 11 hours post-infection cells were washed once in warm medium 199V containing 1% calf serum lacking methionine (Sigma Chemical Co., St. Louis, Mo.) and incubated for an additional hour in 199V methionine-free medium after which cells were overlaid with medium 199V lacking methionine but supplemented with 100 μCi of [³⁵S] methionine (specific activity, >1000 Ci/mmol; Amersham Pharmacia Biotech) per ml and incubated for an additional two hours. The cells were then harvested at 14 hours post-infection, solubilized in lysis buffer [20 mM Tris (pH 7.5), 150 mM NaCl, 1 mM EDTA, 1 mM EGTA, 1% Triton X-100, 2.5 mM sodium pyrophosphate, 1 mM[β-glycerolphosphate, 100 μM sodium orthovanidate, 1 μg leupeptin per ml and 1 mM PMSF], sonicated for 10 seconds, and insoluble material was removed by centrifugation. Total protein from the supernatant was quantified by the Bradford method (BioRad Laboratories, Hercules, Calif.) and 20 μg of equilibrated protein was subjected to electrophoresis in denaturing 12% (vol/vol) polyacrylamide gels, transferred to Polyvinylidene Difluoride membranes (PVDF; Millipore Corporation, Bedford, Mass.) and subjected to autoradiography.

Immunoblotting—Experiments to analyze the accumulation of viral proteins and phosphorylation of ERK, PKR and eIF-2α were performed on whole-cell lysates harvested on ice at either 12 or 14 hours post-infection with lysis buffer, sonicated for 10 seconds, and clarified by centrifugation. Total protein from the supernatant was quantified by the Bradford method and 20 μg of equilibrated protein was subjected to electrophoresis in 12% or 7.5% (vol/vol) denaturing polyacrylamide gels, transferred to PVDF membranes (Millipore Corporation), blocked, and reacted with primary antibody followed by appropriate secondary antibody.

Antibodies—Polyclonal antibodies to the total and phosphorylated forms of PKR (Thr446), eIF-2α (Ser51), and ERK (Thr202/Tyr204) were purchased from Cell Signaling Technology (Beverly, Mass.). Polyclonal antibody to ICP27 was purchased from Santa Cruz Biotechnology (Santa Cruz, Calif.). Monoclonal antibody to Glycoprotein C was purchased from Fitzgerald Industries International, Inc. (Concord, Mass.). Antibodies to Us11 and UL42 were described in refs. 43 and 45, each incorporated herein by reference for the relevant description. Secondary antibodies (Cell Signaling Technology, Beverly, Mass.) were conjugated to horseradish peroxidase. Protein bands were visualized using SuperSignal West Pico Chemiluminescent Substrate (Pierce Biotechnology, Rockford, Ill.).

Inhibitor studies—For experiments employing the known MEK inhibitor, PD98059, HT1080 cells were starved over night in serum-free medium and then exposed to 40 μM of PD98059 (EMD Biosciences, San Diego, Calif.), or DMSO (1:1000 dilution) 6 hours prior to, and during, infection. At 12 hours post-infection, whole cell lysates were created as described above for immunoblotting.

In vitro viral recovery—Cells were exposed to viruses (1 plaque forming unit per cell (PFU/cell)) for 2 hours in serum-free medium at 37° C., after which the supernatant was aspirated and cells were overlaid with 2 ml of DMEM containing 1% calf serum and incubated at 37° C. At 36 hours post-infection, 2 ml of sterile skimmed milk was added to triplicate samples and plates were frozen at −80° C. Frozen cell suspensions were thawed and sonicated three times for 15 seconds each and titered on Vero cells.

HTcaMEK, HTdnMEK xenograft regression studies—HT-dnMEK and HT-caMEK tumor xenografts were established in the right flank of 5- to 6-week-old female, athymic nude mice (Fredrickson Cancer Research Institute, Bethesda, Md.) by injection of 10⁷ cells in 100 μl of warm phosphate-buffered saline. After one week, tumor xenografts grew to approximately 250 mm³ and were randomized to 7 animals per treatment group. Mice were injected intratumorally with 5×10⁷PFU of R3616 using a Hamilton syringe. Tumor xenografts were measured biweekly with calipers and tumor volumes were calculated with the formula (1×w×h)/2, which is derived from the formula for an ellipsoid (™d³)/g (24).

For the studies described in Example 8, tumor xenografts in athymic nude mice were established by hindlimb injection of 5×10⁶ HT-caMEK, HT-dnMEK, Hep3B, or PC-3 tumor cells. At a mean volume of 115-150 mm³, the tumors were treated on days 0 and 5 by administration of R3616 via intratumoral injection of 5×10⁷ PFU or intraperitoneal injection of 10⁶, 10⁷, or 10⁸ PFU of R3616 recombinant virus. Tumor xenografts were measured twice weekly with calipers. Tumor volume was calculated with the formula (1×w×h)/2, derived from the formula for the volume of an ellipsoid (d3/g). Tumor growth was measured at each time point by calculating the ratio of tumor volume (V) to initial tumor volume (V0).

In vivo tumor xenograft regression studies. For the experiments described in Example 9, bilateral tumor xenografts were established in 15 athymic nude mice (Fredrickson Cancer Research Institute, Bethesda, Md.) by subcutaneous injection into each hindlimb of 5×10⁶ PC-3 tumor cells in 100 μL of PBS. Eight days after inoculation, with initial tumor volumes of 260±41 mm³, mice were either treated by bilateral intratumoral injection of 1×10⁷ plaque-forming units (pfu) of R2660 (10 mice) or left untreated (5 mice). On day 1, 24 hours following viral injection, the right hindlimb tumors of all 15 mice were exposed to ionizing radiation (IR) at a dose of 20 Gy using a Philips orthovoltage x-ray generator (Philips Medical Systems, Bothell, Wash.) operating at 250 kV and 15 mA. Mice were restrained in plexiglass shields and all areas except the tumor-bearing hindlimb were shielded with lead. Xenografts were measured on days 0, 5, 7, 9, 12, and 15 with calipers. Tumor volume was calculated using the formula V=(l×w×h)/2, derived from the formula for the volume of an ellipsoid, πd³/6. Tumor growth was measured at each time point by calculating the ratio of tumor volume V to initial tumor volume V₀.

Bioluminescence Imaging—HT-dnMEK and HT-caMEK tumor xenografts were established in the left and right hind limbs, respectively, of athymic nude mice by injection of 1×10′ cells in 100 μl of warm phosphate-buffered saline. All animal studies were performed in accordance with The University of Chicago Animal Care and Use Committee standards. Once tumors grew to an average volume of 350 mm³, 9×10⁸ PFU of virus R2636 in a total volume of 100 μl were injected intraperitoneally (IP) using a 30-gauge needle. At 5 days after IP injection, imaging of firefly luciferase in mice was performed on a charge-coupled device camera (Roper Scientific Photometrics, Tucson, Ariz.). Animals were injected IP with 15 mg/kg body weight with D-luciferin (Biotium, Hayward, Calif.). After 5 minutes, animals were anesthetized with IP injection of ketamine (75 mg/kg) and xylazine (5 mg/kg) for imaging, which was performed 10 minutes after the injection of D-luciferin.

Again for the studies described in Example 8, HT-dnMEK and HT-caMEK xenografts were established in the right hindlimb of athymic nude mice by injection of 5×10⁶ cells. At initial tumor volumes of 175±60 mm³ for HT-caMEK and 131±22 mm³ for HT-dnMEK, mice were injected with either intratumoral (5×10⁷ PFU) or intraperitoneal (IP) (10⁸ PFU) R2636. Animals were imaged on days 1, 3, 8, 12, and 22 following viral injection. Imaging was performed on a charge-coupled device camera (Roper Scientific Photometrics, Tucson, Ariz.). On days of imaging, animals were injected IP with D-luciferin (Biotium, Hayward, Calif.) at a dose of 15 mg/kg of body weight. After 5 minutes, animals were anesthetized with IP injection of ketamine (75 mg/kg) and xylazine (5 mg/kg) for imaging, which was performed 10 minutes after injection of D-luciferin.

Quantification of bioluminescence imaging data—The relative intensity of transmitted light from animals infected with virus R2636 are represented as pseudocolor images with intensity ranging from low (blue) to high (red). Gray-scale images were superimposed on the pseudocolor images using MetaMorph image analysis software (Fryer Company, Huntley, Ill.). Data for total photon flux were calculated using area under the curve analysis (MetaMorph).

### EXAMPLE 2

### Correlation of γ₁34.5 Deficient HSV Replication and MEK Phenotype of Host Cells

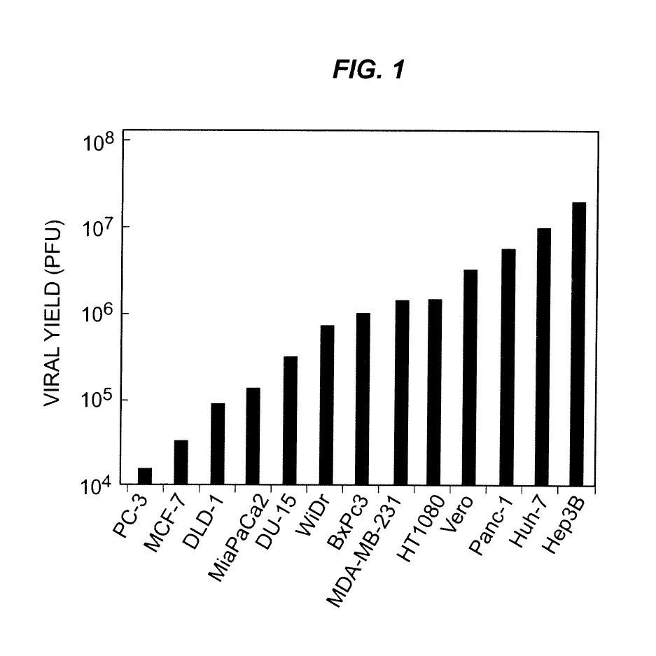

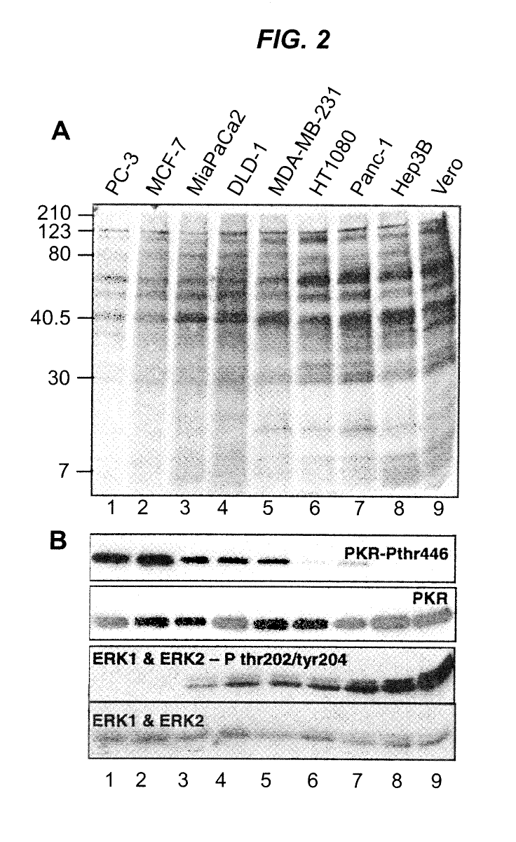

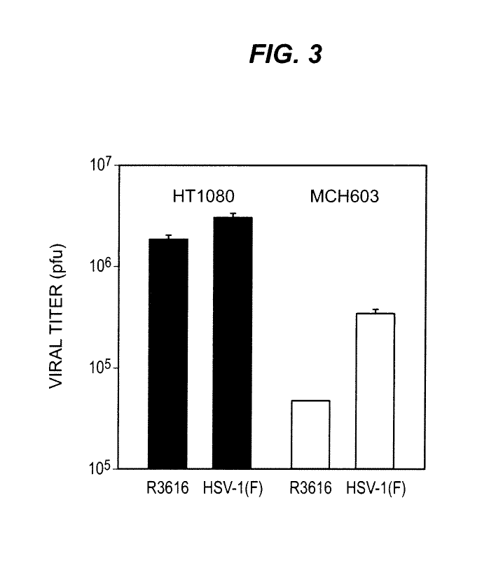

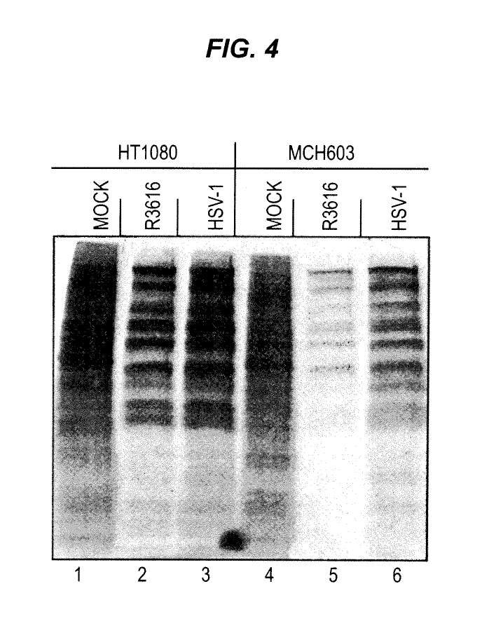

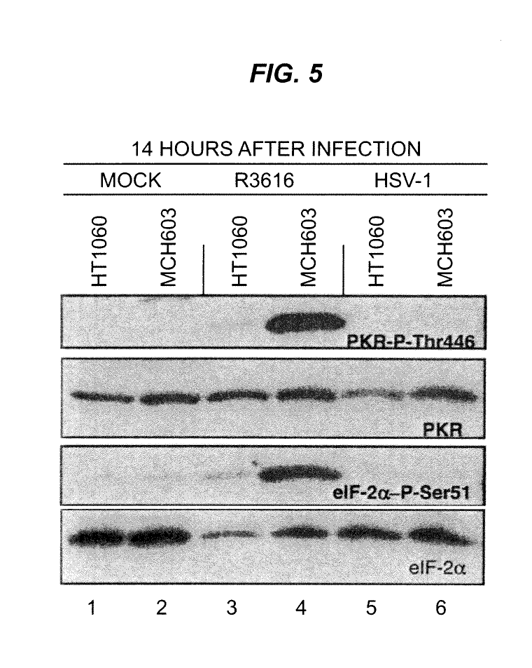

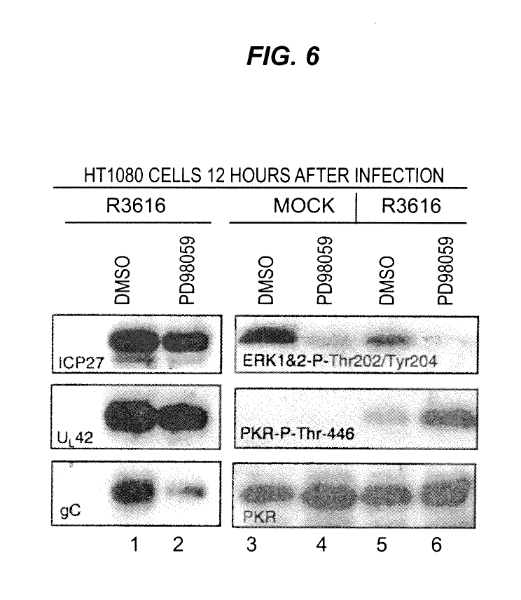

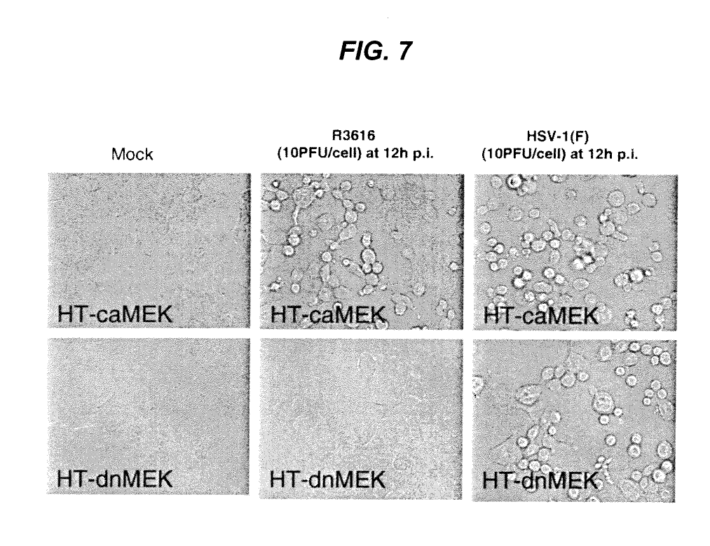

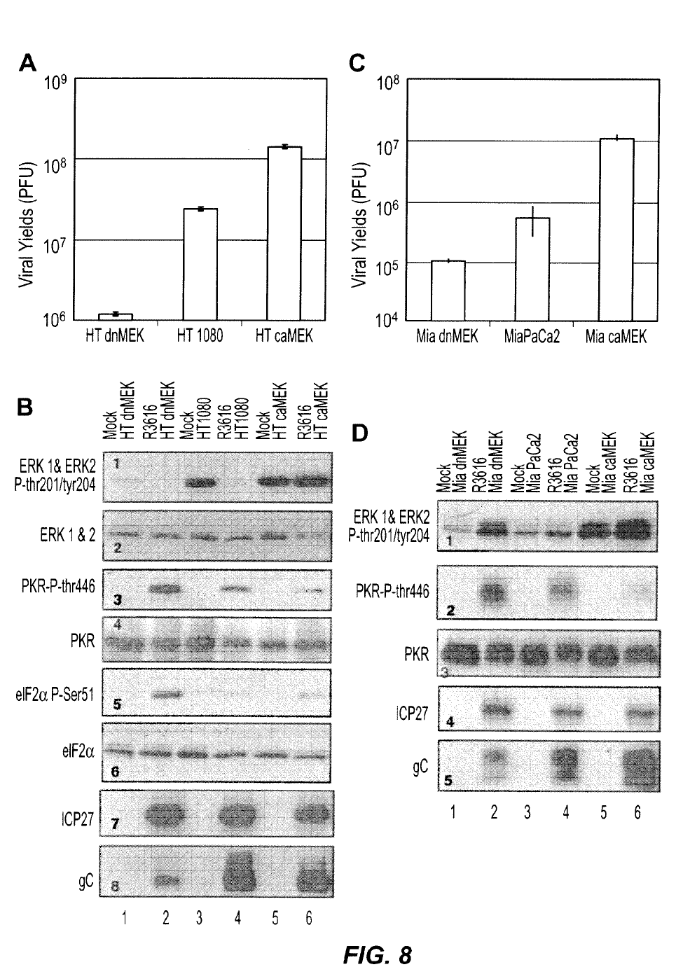

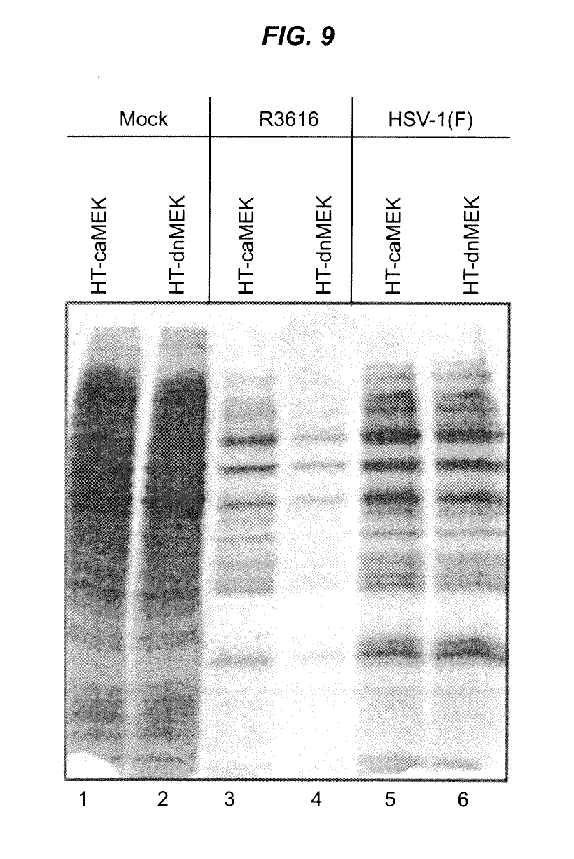

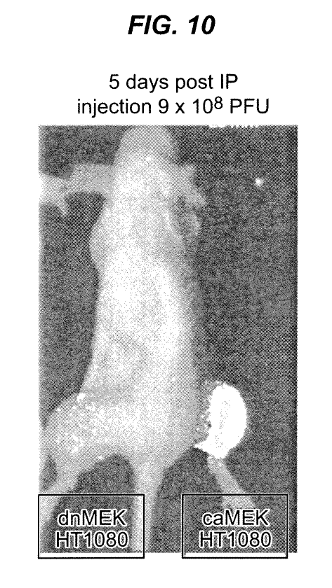

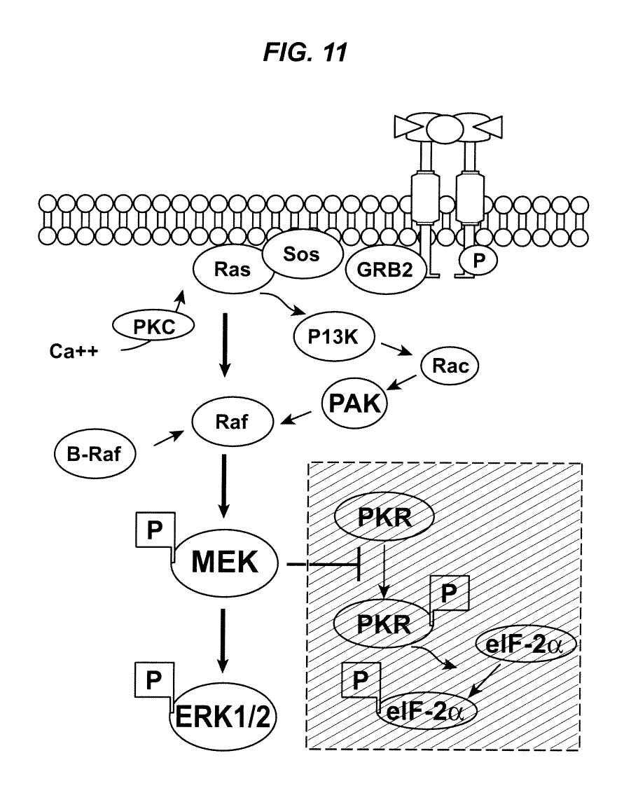

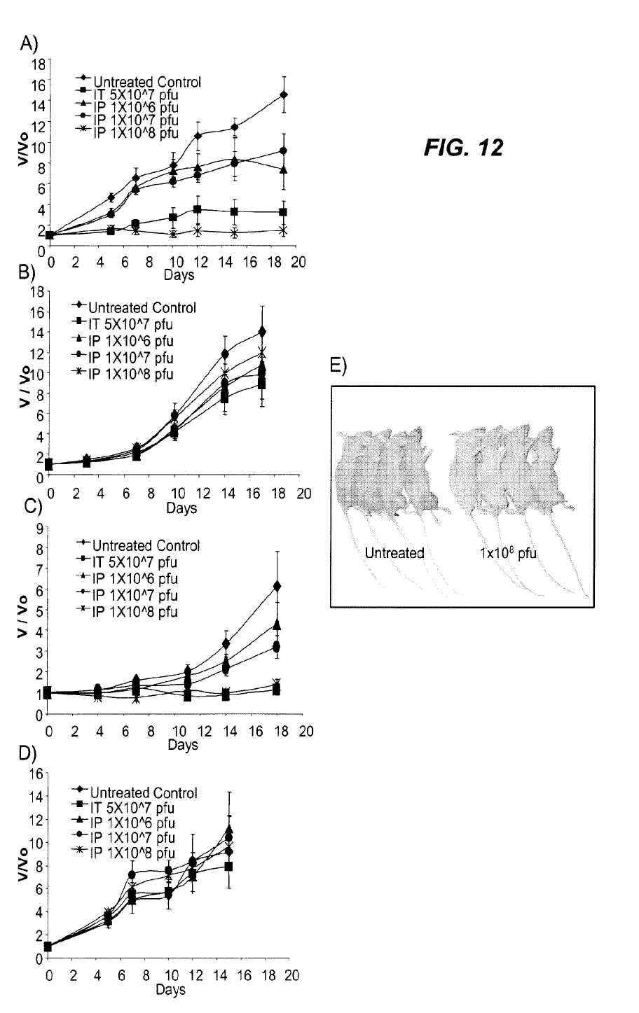

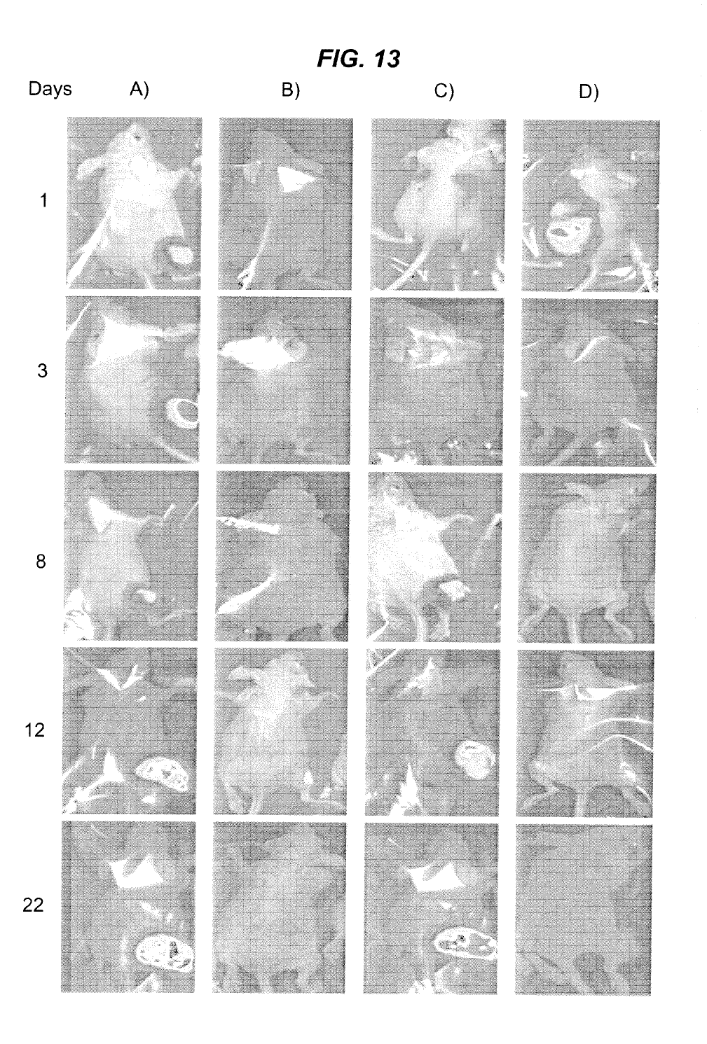

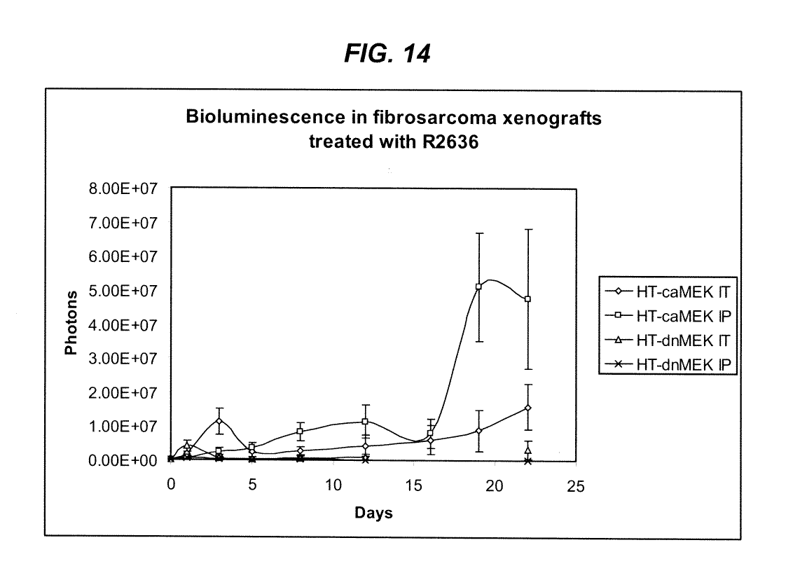

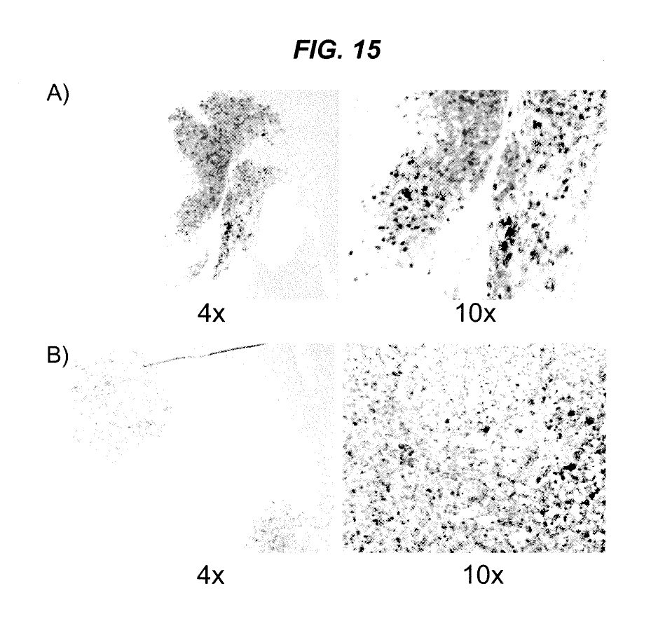

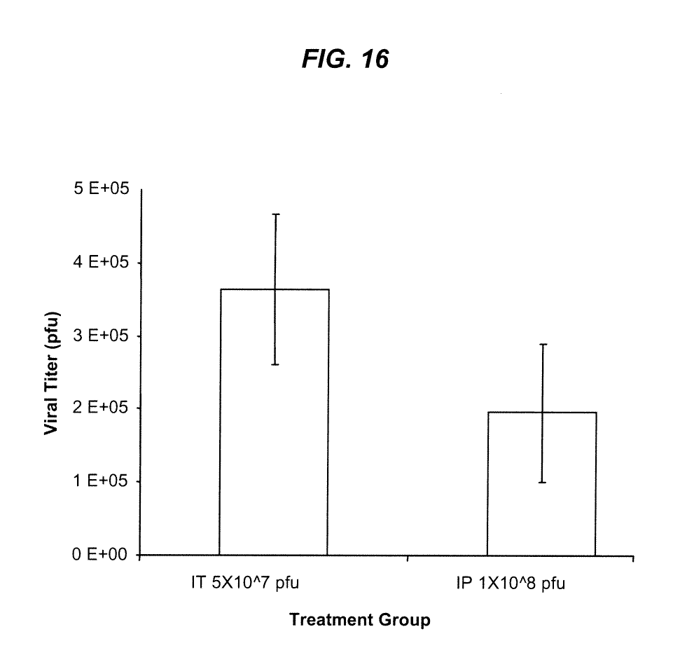

The replication of R3616 (Δγ₁34.5) mutant virus in human tumor cell lines is cell line dependent and correlates with constitutive activation of MEK. Replicate cultures of 13 cell lines derived from human tumors were exposed to R3616 (1 PFU/cell). The cells were harvested at 36 hours post-infection and viral yields were measured by plaque assays on Vero cell monolayers. As shown in FIG. 1, the yields of R3616 mutant virus were variable, ranging from 1×10⁴ to 3×10⁷ PFU/ml. To determine whether the variability in virus yields was reflected in the accumulation of viral proteins, cultures of human tumor cell lines were exposed to R3616 (10 PFU/cell). Vero cells were included as an example of a nonmalignant cell type that supports replication of γ₁34.5 deficient viruses. At 11 hours post-infection, the cells were rinsed, starved of methionine for one hour, and then incubated in methionine-free medium supplemented with 100 μCi of [³⁵S] methionine per ml for two additional hours. At 14 hours post-infection, 20 μg of equilibrated protein lysates were electrophoretically separated in denaturing polyacrylamide gels, transferred to a PVDF membrane, and exposed to autoradiography film. As shown in FIG. 2, panel A, the accumulation of viral proteins was reduced in cell lines that restricted viral replication compared to cell lines where viral yields were abundant.

To correlate the differences in the accumulation of viral proteins with the activation of PKR, replicate cultures of cell lines shown in FIG. 2, panel A, were exposed to R3616 (10 PFU/cell) for 14 hours. Lysates were harvested and 20 μg of equilibrated whole-cell lysate were electrophoretically separated in denaturing polyacrylamide gels, transferred to PVDF membranes, and reacted with antibody specific for the phosphorylated form of PKR in which Thr446 is phosphorylated. As shown in the upper panels of FIG. 2, B, PKR phosphorylation was elevated in the cell lines which yielded reduced viral protein accumulation (e.g., PC-3, MCF-7) and lowest in cell lines that exhibited increased levels of viral protein accumulation (e.g., HT1080, Panc-1, Hep-3B, Vero), while total PKR levels were similar.

The presence of known activating mutations within the commonly mutated oncogenic (K-, H-, N-Ras) isoforms of Ras, however, did not directly correlate with the observed differences in viral recovery from the representative cell lines identified in FIG. 1. Therefore, the constitutive activity of the downstream effectors of Ras, MEK and its substrate, ERK, which when inhibited results in the loss of the inhibitory functions of Ras on PKR (19), were examined. To determine endogenous constitutive MEK activity, uninfected cells were plated to confluence, serum-starved for 12 hours, and then immunoblotted for the phosphorylated and total forms of the MEK substrate, p42 and p44 MAPK (ERK2 and ERK1, respectively), see FIG. 2 B, lower panels. Cell lines that demonstrated increased protein synthesis and suppressed PKR activation following infection with mutant R3616 demonstrated elevated baseline levels of ERK phosphorylation. In contrast, cancer cell lines that demonstrated PKR activation, inhibited protein synthesis, and decreased viral recovery following infection with R3616 demonstrated decreased or undetectable levels of ERK phosphorylation.

### EXAMPLE 3

### N-Ras Mutation Enabled Efficient Replication of R3616 Mutant Virus in Human Fibrosarcoma Cells

To test the hypothesis that Ras/Raf/MEK/MAPK (ERK) signaling suppresses PKR function, replication of R3616 mutant virus in two human fibrosarcoma cell lines that differ only by the expression of an oncogenic mutant allele of N-Ras were measured. HT1080 cells contain an endogenous activating mutant allele of N-Ras, whereas the MCH603 cell line, a variant of HT1080 cells in which the mutant allele has been deleted, contains only wild-t e N-Ras (40). Activated MEK is a prerequisite for the Ras-dependent aggressive tumorigenic phenotype of HT1080 cells and the two cell lines differed dramatically in the constitutive levels of MEK activation, as well as in activation levels of downstream members of the Ras signaling pathway (21). The viral yields of HSV-1(F) and R3616 (1 PFU/cell) at 36 hours post-infection are shown in FIG. 3. The results led to two significant observations. First, the yield of HSV-1(F) from the MCH603 cell line was approximately 10-fold lower than that obtained from HT1080 cells (3.1×10⁷compared to 3.5×10⁶), respectively. Second, the yield of R3616 mutant virus in HT1080 cells was similar to that of wild-type virus (1.8×10⁷ versus 3.1×10⁷), indicating that γ₁34.5 function was not necessary during the course of infection in this cell line. In contrast, the yield of R3616 mutant virus was approximately 10-fold lower than that of wild-type virus in MCH603 cells, with yields of 4.8×10⁵compared to 3.5×10⁶, respectively. Therefore, the presence of an activating N-Ras mutation enhanced the replication of both wild-type and mutant virus and that effect was greater on the virus lacking a functional γ₁34.5 gene.

To determine whether virus yields correlate with overall levels of the accumulation of viral proteins, replicate cultures of HT1080 or MCH603 cells were mock-infected or exposed to viruses R3616 or HSV-1(F) (10 PFU/cell). At 11 hours post-infection, the cells were rinsed, starved of methionine for one hour, and then supplemented with 100 μCi/ml of [³⁵SJ methionine for two additional hours. At 14 hours post-infection, 20 μg of equilibrated protein lysates were electrophoretically separated in denaturing polyacrylamide gels, transferred to PVDF membranes and subjected to autoradiography. The results shown in FIG. 4 are congruent with viral yields obtained from the two cell lines. Specifically, the abundance of labeled proteins in MCH603 cells infected with wild-type virus was significantly greater than that observed in the same cells infected with R3616 mutant virus, with both of the MCH603 protein yields being lower than the amounts of proteins accumulating in HT1080 cells infected with either mutant or wild-type virus.

Lastly, the correlations of each of (1) virus yields and (2) viral protein levels accumulating in infected cells with each of (3) PKR activation and (4) phosphorylation of eIF-2α, were assessed. Electrophoretically separated proteins of lysates from cells infected with R3616 and HSV-1(F) (10 PFU/cell) were harvested at 14 hours post-infection and probed with antibodies to PKR and the phosphorylated forms of PKR (P-Thr446) and eIF2α (P-Ser51). As shown in FIG. 5, both PKR and eIF-2α were phosphorylated in MCH603 cells infected with R3616 mutant virus. In contrast, only trace amounts of phosphorylated PKR and eIF-2α were detected in infected HT1080 cells.

### EXAMPLE 4

### Inhibition of MEK by PD98059 Resulted in Increased Levels of PKR Phosphorylation, Decreased Viral Protein Accumulation and Diminished Replication of Mutant Virus R3616

To determine if MEK mediates the observed mutant Ras-dependent suppression of PKR activation and resultant accumulation of viral proteins in HT1080 cells infected with mutant virus R3616, the relative expressions of representative α (ICP27), β (U_{L}42) and γ2(glycoprotein C) viral proteins in cells treated with a specific chemical inhibitor of MEK-1 (PD98059) were compared. Replicate cultures of HT1080 cells were serum-starved overnight prior to exposure to equal volumes of DMSO or PD98059 (40 μM) for 6 hours prior to infection with R3616 mutant virus (10 PFU/cell). DMSO or drug treatment was then continued until the cells were harvested at 12 hours post-infection. The cells were then lysed and the lysates were subjected to electrophoresis in denaturing polyacrylamide gels, followed by transferring to PVDF membranes and reacting with antibody to ICP27, UL42, or gC. As shown in FIG. 6, panel A, treatment with PD98059 had a slight effect on the accumulation of ICP27 and UL42 proteins but a very dramatic decrease in the amounts of gC that accumulated in }{T1080 cells infected with R3616. To test whether the decrease in the accumulation of gC correlated with activation of PKR, the electrophoretically separated lysates were also probed with antibody to the auto-phosphorylated form of PKR (P-Thr446). The presence of PD98059 prior to, and during, infection with R3616 increased the amount of activated PKR in HT1080 cells (FIG. 6, panel B).

These results are consistent with the earlier report that in wild-type virus-infected cells, PKR activation is concurrent with the onset of viral DNA synthesis and enhanced transcription of late genes. In R3616 mutant virus-infected cells, the phosphorylation of eIF-2α by PKR causes a significant reduction of viral proteins whose accumulation is dependent on viral DNA synthesis (14). In contrast, viral proteins whose synthesis is not dependent on the onset of viral DNA synthesis (e.g., ICP27, UL42 protein) were minimally affected by the activation of PKR.

Finally, to determine if MEK inhibition affects viral replication, DMSO or PD98059 (40 μM) was added to replicate cultures of HT1080 cells 6 hours prior to, and during, infection with R3616 (1 PFU/cell). The cells were harvested at 36 hours post-infection and viral yields were measured by plaque assays on Vero cell monolayers. In the presence of PD98059, the yield of R3616 mutant virus was approximately 15-fold lower than in the presence of DMSO (4.14×10⁶ compared to 1.67×10⁵PFU/ml).

### EXAMPLE 5

Viral Activation of PKR by Mutant R3616 is Suppressed in Tumor Cell Lines that Overexpressed Constitutively Active MEK, while Expression of Dominant Negative MEK Increased PKR Activation and Restricted R3616 Viral Replication

To study the potential relationship between MEK kinase activity and PKR activation in R3616-infected cancer cells, cell lines were created that stably express either a constitutively activated mutant of MEK (caMEK) or a dominant negative mutant of MEK (dnMEK) from two tumor cell lines that differ dramatically in the magnitude of endogenous MEK activity and the ability to support R3616 viral replication. MEK is constitutively active in the HT1080 human fibrosarcoma cell line. This cell line, as shown in FIG. 1-3, is also highly permissive to R3616 viral replication and demonstrates suppressed viral activation of PKR. In contrast, the MiaPaCa2 cell line, which is derived from a patient with poorly differentiated malignant pancreatic adenocarcinoma, contains oncogenic K-Ras mutations in both alleles but demonstrates nearly undetectable levels of constitutively active MEK (50). The MiaPaCa2 cell line severely restricts R3616 viral replication and demonstrates robust PKR activation during R3616 viral infection.

Mutant cDNAs of human MEK-1 containing mutations in serine codons at amino acid positions 218 and 222 that resulted in codons encoding negatively charged aspartate residues have been generated. These mutations mimic the effect of phosphorylation at positions 218 and 222, resulting in constitutive activation of MEK-1 (MAPK—kinase) function (27). In contrast, alanine substitutions at the same residues functionally block phosphorylation by upstream MAPK-kinase-kinases (MAPKKKs), resulting in down-regulation of endogenous MAPK activity (34). Plasmids, designated pNC84 and pNC92, containing the respective N-terminal FLAG-tagged [Asp218, Asp222 MEK-1] or [Ala218 and Ala222 MEK-1] cDNAs under the transcriptional control of a CMV promoter and the neomycin resistance gene, were used to select for G418 resistance, FLAG-MEK expressing clonal transfectants as described in Example 1.

As shown in FIG. 7, when the mutant MEK-expressing HT1080 stable cell lines were infected with mutant R3616 (10 PFU/cell), there were appreciable differences in cytopathic effects (CPE). HT-caMEK cells exhibited CPE at 12 hours post-infection while HT-dnMEK-expressing cells did not. Both cell lines, however, exhibited CPE upon infection with HSV-1(F) (10 PFU/cell). Next, viral recoveries were compared from the stable transfectants generated from HT1080 and MiaPaCa2 cells after exposure of the cells to 1 PFU of R3616 virus per cell. There was a greater than 200-fold increase in viral titer in R3616-infected caMEK cells compared with dnMEK cells, i.e., 1.18×10⁶ compared to 1.46×10⁸ PFU/ml for the HT1080 transfectants (caMEK v. dnMEK, respectively), and 1.05×10⁵ compared to 1.10×10⁷PFU/m1 for the MiaPaCa2 transfectants (caMEK v. dnMEK, respectively). See FIG. 8, panels A and C.

Lastly, three series of experiments were done to determine whether the enhancement of replication of the R3616 mutant virus in caMEK cells correlated with increased accumulation of viral proteins and inhibition of PKR activation. In the first experiment, dnMEK- and caMEK-expressing cell lines and their respective parent cell lines were exposed to 10 PFU per cell of mutant virus R3616 (FIG. 8). The cells were harvested 12 hours post-infection, solubilized, subjected to electrophoresis in denaturing polyacrylamide gels and reacted with antibodies to PKR, eIF-2α and the phosphorylated forms of PKR (P-Thr446) and eIF-2α (P-Ser51), respectively. Baseline differential MEK activities in uninfected dnMEK- and caMEK-expressing cells and the parental cell lines were established by immunoblotting whole-cell lysates with antibody to ERK1/ERK2 and the phosphorylated form of ERK1/ERK2 (P-Thr202 and P-Tyr204, respectively), see Panels B-1 and D-1 of FIG. 8. As shown (Panels B-3 and D-2 of FIG. 8), levels of phosphorylated PKR and eIF-2α were higher in dnMEK-expressing lines infected with the R3616 mutant virus as compared with the parental cell line or the caMEK-expressing cell lines. Conversely, activated PKR was nearly undetectable in caMEK-expressing cells infected with the R3616 mutant virus.

In the second series of experiments, electrophoretically separated lysates of caMEK- or dnMEK-expressing cell lines that had been infected with the R3616 mutant virus and processed as described above were reacted with antibody to a (ICP27) and γ2 (glycoprotein C) proteins. As shown in Panel B-7 and Panel D-4 of FIG. 8, the accumulation of ICP27 was similar in both the stably transfected mutant cell lines and the parental cell lines, suggesting that the expression of MEK-1 mutants did not significantly affect the accumulation of ICP27, a protein expressed prior to the onset of viral DNA synthesis. However, consistent with the result shown in FIG. 6 with chemical inhibition of MEK, the accumulation of gC was markedly decreased in dnMEK-expressing cell lines at 12 hours post-infection, compared with the parent or caMEK-expressing stable cells (Panel B-8 and Panel D-5 of FIG. 8).

Lastly, caMEK- or dnMEK-over-expressing HT1080 cell lines were exposed to 10 PFU of virus HSV-1(F) or mutant R3616. At 11 hours post-infection, the cells were rinsed, starved of methionine for one hour, and then supplemented with 100 μCi/ml of [³⁵S] methionine for two additional hours. At 14 hours post-infection, 20 μg of equilibrated protein lysates were electrophoretically separated in denaturing polyacrylamide gels, transferred to PVDF membranes, and exposed to autoradiography film. As shown in FIG. 9, the accumulation of labeled proteins was similar in HT-caMEK (lane 5) and HT-dnMEK (lane 6) cells during infection with HSV-1(F). In contrast, the accumulation of labeled proteins in HT-dnMEK cells (lane 4) was diminished compared with HT-caMEK cells (lane 3) infected with the R3616 mutant virus.

### EXAMPLE 6

Intratumoral Inoculation of R3616 Mutant Virus Resulted in Tumor Regression in Tumors Expressing caMEK but Not in Tumors Expressing dnMEK

To determine if differential replication correlated with a reduction of tumor size, we measured tumor volumes of untreated and R3616-treated HT-caMEK and HT-dnMEK tumor xenografts. HT-dnMEK and HT-caMEK tumor xenografts were grown to an average volume of 250 mm³ and injected with a single dose of 5×10⁷ PFU of R3616 or buffer on day 0. At 31 days after infection by the R3616 mutant virus, only 1/7 animals had a palpable HT-caMEK tumor (100 mm³), in comparison to untreated HT-caMEK tumors, which averaged (4300+/−730 mm³ (standard error of the mean (SEM))). In contrast, all (7/7) of the HT-dnMEK tumors were palpable, with an average tumor volume of (830+/−SEM 210 mm³) and untreated HT-dnMEK tumor volumes averaged (4000+/−SEM 660 mm³).

### EXAMPLE 7

Systemic Administration of a Recombinant Virus R2636 Expressing the gC-Luc Construct Targeted Tumor Tissue Over-Expressing Constitutively Active MEK

To determine whether differential MEK activity confers tumor-selective viral replication upon systemic delivery of a γ₁34.5-deficient virus, bilateral hindlimb tumor xenografts were grown by injecting the left and right hindlimbs of athymic nude mice with 5×10⁶ cells of the HT-dnMEK and HTcaMEK cell lines, respectively. In order to image viral replication in vivo, mutant HSV R2636 was used, which is γ₁34.5-deficient virus that expresses the firefly luciferase gene under the transcriptional control of the HSV-1 gC-promoter, a representative γ promoter (37). In tissue that restricts viral replication, the accumulation of the firefly luciferase gene product expressed with the kinetics of a γ gene, such as gC, would be decreased over successive replicative cycles by PKR-mediated shutoff of protein synthesis. However, a Δγ₁34.5 mutant virus-infected, caMEK-xenografted, tumor cells, which support viral replication and gC expression, was expected to support R2636 replication and express gC-luciferase enzyme activity. At 5 days after IP delivery of R2636, bioluminescence localized to the right hindlimb, which corresponded to the caMEK-xenografted tumor (3,692 photons/mm²/sec) while the dnMEK tumor xenograft demonstrated 95-fold less photon expression (39 photons/mm²/sec). Also, there was no detectable bioluminescence outside of the caMEK-expressing tumors by 5 days post-IP injection (FIG. 10).

### EXAMPLE 8

### Comparative Study of Intratumoral and Systemic Delivery of Virus

A series of experiments was designed to compare the intratumoral and systemic delivery of genetically engineered virus on tumor xenografts derived by injection of isogenic tumor cells differing with respect to ectopically-expressed MEK activity. General experimental techniques employed have been described in Example 1, above. Tumor xenografts were established by injecting 5×10⁶ HT-caMEK or HT-dnMEK tumor cells into the hindlimbs of athymic nude mice. At a mean volume of 115±13 mm³, the tumors were treated on days 0 and 5 by administration of R3616 via intratumoral injection of 5×10⁷ PFU or intraperitoneal injection of 10⁶, 10⁷, or 10⁸ PFU of R3616 recombinant virus. Tumor xenografts were measured twice weekly with calipers. Tumor volume was calculated with the formula (l×w×h)/2, derived from the formula for the volume of an ellipsoid. Tumor growth was measured at each time point from day 0 to day 19 by calculating the ratio of tumor volume (V) to initial tumor volume (V₀). The results of these experiments are shown in FIG. 12. In the HT-caMEK xenografts (FIG. 12A), intraperitoneal treatment with 2×10⁶, 2×10⁷, or 2×10⁸ PFU of R3616, resulted in a significant dose-dependent tumor response by 19 days (V/V₀ of 9.1±1.9, 7.3±1.6, and 1.5±0.6, respectively) compared to untreated HT-caMEK controls (V/V₀ of 14.5±1.7) (p=0.0221, 0.0371, and 0.0007, respectively). In HT-dnMEK xenografts (FIG. 12B), no significant effect on tumor growth was seen by day 15 with intraperitoneal administration of 2×10⁶, 2×10⁷, or 2×10⁸ PFU of R3616 (V/V₀ of 11.2±1.9, 10.4±1.6, and 9.6±0.6, respectively) compared to untreated HT-dnMEK controls (V/V₀ of 9.1±3.1) (p=0.46, 0.35, 0.14, respectively). Intratumoral administration of 10⁸ PFU of R3616 in HT-caMEK xenografts resulted in a significant anti-tumor effect with a V/V₀ of 3.2±1.1 by day 19 (p=0.0020). Intratumoral administration of 10⁸ PFU of R3616 in HT-dnMEK xenografts did not demonstrate a significant anti-tumor effect with V/V₀ of 7.9±1.1 by day 15 (p=0.36). Thus, tumor xenografts genetically engineered to express constitutively active MEK were susceptible to oncolysis following systemic delivery by intraperitoneal injection of R3616, while xenografts engineered to express dominant-negative MEK activity were resistant to R3616 oncolysis.

In the second set of experiments, xenografts were established in the hindlimbs of athymic nude mice consisting of Hep3B cells, a human hepatoma cell line, and PC-3 cells, a human prostate cancer cell line. As reported earlier, Hep3B expressed high MEK activity whereas the PC-3 cells expressed almost no MEK activity (Smith et al., J Virol 80:1110-1120 (2006)). Hep3B and PC-3 xenografts were established in nude mice by hindlimb injection of 5×10⁶ cells per animal. Hep3B and PC-3 xenografts were grown to an average volume of 150±4 mm³, and then treated on days 0 and 5 with either intratumoral injection of 5×10⁷ PFU of R3616 or intraperitoneal injection of 10⁶, 10⁷, or 10⁸ PFU of R3616. Hep3B xenografts (FIG. 12C) demonstrated a dose-dependent effect with intraperitoneal administration of 2×10⁶, 2×10⁷, and 2×10⁸PFU of R3616 which resulted in V/V₀ of 4.3±1.0, 3.2±0.5, and 1.4±0.3 at 18 days compared to untreated Hep3B controls which reached a mean V/V₀ of 6.1±1 (p=0.2050, 0.0858, and 0.0135, respectively).

In PC-3 xenografts (FIG. 12D) there was no significant difference between intraperitoneal doses of 2×10⁶, 2×10⁷, and 2×10⁸ PFU of R3616 (p=A2327, 0.0882, 0.2970, respectively) and untreated control PC-3 xenografts by day 17. Intratumoral administration of 10⁸ PFU of R3616 into Hep3B xenografts (FIG. 12C) resulted in a V/V₀ of 1.1±0.2 (p=0.0130) by day 18. In PC-3 xenografts, intratumoral administration of 10⁸ PFU of R3616 did not result in a significant antitumor effect with a V/V₀ of 8.9±2.2 (p=0.102) (FIG. 12D). These results demonstrated that tumor regrowth studies with natively high (Hep3B) and low (PC-3) MEK activity tumors were similar to the results obtained with tumors genetically engineered to express constitutively active or dominant-negative MEK activity.

Luciferase imaging demonstrated increased viral replication which localized to HT-caMEK tumors compared to attenuated viral replication in HT-dnMEK tumors. R2636 is a γ₁34.5-deficient virus constructed from the R3616 backbone that expresses the firefly luciferase gene under the control of the late HSV-1 gC promoter. Using R2636, in vivo imaging of viral replication was obtained. Detectable luciferase expression in tissues connotes active viral replication because gC-driven expression marks the expression of late viral structural genes. Hindlimb xenografts were established in nude mice by the injection of 5×10⁶ cells of the fibrosarcoma cell lines HT-caMEK or HT-dnMEK. At initial tumor volumes of 175±60 mm³ for HT-caMEK and 131±22 mm³ for HT-dnMEK, mice were injected with either intratumoral (5×10⁷ PFU) or intraperitoneal (10⁸ PFU) R2636. Animals were imaged on days 1, 3, 8, 12, and 22 following viral injection.

In HT-caMEK xenografts that received intratumoral injections (FIG. 13A), an increase in luminescence remained localized to the hindlimb only. In HT-dnMEK xenografts injected intratumorally, luminescence reached a plateau early in the study and demonstrated much lower activity than their HT-caMEK counterparts injected intratumorally (FIG. 13B). HT-caMEK tumor-bearing mice (FIG. 13C) that received intraperitoneal R2636 demonstrated an increase in luminescence in the abdominal cavity (in the liver or spleen) on day 1 that disappeared by day 3 and remained absent up to the conclusion of the study at day 22, while a steady increase in luminescence was observed in the hindlimb bearing xenografted tumors. HT-dnMEK tumor-bearing mice treated by intraperitoneal R2636 (FIG. 13D) demonstrated a similar increase in luminescence in the abdominal cavity, liver and spleen, on day 1 and day 3, which abated by day 8 and remained absent up to the conclusion of the study on day 22, with no localization to the hindlimb xenografts. Luminescence was measured and relative intensity quantified as total photon flux (FIG. 14). HT-dnMEK tumors treated with either intratumoral or intraperitoneal R2636 failed to demonstrate significantly increased luminescence above the baseline luminescence measured in untreated HT-dnMEK control tumors.

To study intratumoral distribution of R3616 in HT-caMEK tumors following IT or IP injection, xenografts were harvested 5 days after treatment with either 5×10⁷ PFU of intratumoral or 10⁸ PFU of intraperitoneal R3616. Immunohistochemistry (IHC) for HSV-1 antigen in HT-caMEK xenografts injected intratumorally demonstrated viral replication along the needle track. (FIG. 15A). In contrast, HT-caMEK xenografts treated by intraperitoneal injection demonstrated a more diffuse pattern of viral distribution with multiple foci of viral replication throughout the tumors. (FIG. 15B). No HSV-1 antigens were detected by IHC in HT-dnMEK xenografts 5 days following intratumoral or intraperitoneal injection. To examine recovery of R3616 from HT-caMEK tumors following treatment with either intratumoral or intraperitoneal R3616, HT-caMEK xenografts were harvested 5 days post treatment with either intratumoral 5×10⁷ PFU or intraperitoneal 10⁸ PFU of R3616. Viral titers from homogenized samples were determined by standard plaque formation assays on Vero cell monolayers. Intratumoral administration of 5×10⁷ PFU of R3616 yielded a titer of 4×10⁵±1×10⁵PFU. Intraperitoneal administration of 10⁸ PFU of R3616 yielded a comparable titer of 2×10⁵±1×10⁵ PFU (FIG. 16). No detectable levels of R3616 were recovered from HT-dnMEK xenografts treated with either intraperitoneal 10⁷ or 10⁶ PFU at day 5.

Systemic delivery of R3616 was explored because of the observation that MEK activity suppressed PKR following tumor cell infection with R3616 and thereby increased viral recovery from tumors injected with the virus. Salient observations on the systemic administration of HSV-1 arising from the studies reported herein are: i) R3616 demonstrated greater oncolytic activity in xenografted flank tumors with high levels of active MEK as compared with tumors that expressed lower levels of active MEK. This finding held true in human tumors genetically engineered to express constitutively active MEK, as well as tumors that natively express high MEK activity. ii) The superior oncolytic effects of R3616 in high MEK-activity tumors are corroborated by in vivo imaging studies with R2636, a γ₁34.5 mutant based on the R3616 backbone in which the late viral promoter for gC drives luciferase expression. In vivo imaging with R2636 demonstrated that systemic administration permitted γ₁34.5 mutant virus localization to constitutively active MEK tumors with subsequent intratumoral viral replication. In contrast, in dominant-negative MEK xenografts, R2636 replication was diminished and systemic administration of R2636 did not lead to persistent intratumoral viral replication. iii) Although equal amounts of virus were recovered from caMEK-expressing tumors five days following intraperitoneal administration as compared with intratumoral administration, the kinetics of viral proliferation differed, as reflected by quantified bioluminescence imaging.

Although, intraperitoneal delivery of virus required a two-fold higher dose compared to intratumoral injection to achieve the same oncolytic efficacy, the data reported herein establish that systemic delivery of R3616 effectively treated metastases from these tumors. Also, assays of MEK activation and other kinases in tumors is expected to allow for individualized targeted therapy with R3616 or similar viruses, i.e., γ₁34.5 deficient HSV, including γ₁34.5 HSV. Notably, anti-HSV-1 immune activity has not been reported to limit the use of Δ₁34.5 mutants in human trials to date. The data disclosed herein indicate that γ₁34.5 mutant viruses will be useful in the treatment of disseminated metastatic disease. The following references, numbered 1-36 and 38-50, have been cited throughout this disclosure and are hereby incorporated by reference in their entireties.

### EXAMPLE 9

### Therapeutic HSV Having a Broad Cancer Cell Host Range

The γ₁34.5 mutants of HSV-1 have proven to be safe for human intracerebral administration (Shah et al., J. Neuro-Oncol. 6:203-226 (2003)) and have been effective in destruction of malignant glioma cells in a small fraction of patients tested to date. The shortcoming of the mutant viruses for cancer treatment stems from the observation that the virus replicates only in cells in which the protein kinase R (PKR) pathway is damaged or suppressed (Smith et al., J. Virol. 80:1110-1120 (2006)). Activation of the PKR pathway results in the phosphorylation of the α subunit of the translation initiation factor eIF-2, induction of interferon, and shutoff of protein synthesis (Chou et al., Proc. Nat. Acad. Sci. (USA) 92: 10,516-10,520 (1995)). In most malignant glioma tumors, PKR is either active or capable of being activated after infection with HSV-1. The activation occurs relatively early in infection and as a consequence viral genes are not expressed. Disclosed herein are therapeutic viruses that overcome the tumor genotype restriction to enable γ₁34.5 mutants of HSV-1 to replicate and destroy malignant glioma cells regardless of the status of the PKR pathway.

One feature of the therapeutic viruses disclosed herein is the use of an inducible expression control element (e.g., a promoter) operatively linked to a nucleic acid, e.g., a coding region, that, when expressed, leads to activation or an increase in the activation of the MEK pathway. One contemplated class of inducible expression control element is the inducible promoter. One type of inducible promoter that provided unexpectedly tight control of gene expression was the radioinducible promoter. An exemplary radioinducible promoter is the promoter for HSV gC. Exposure of tumors infected with γ₁34.5 mutants of HSV-1 to ionizing radiation (IR) resulted in earlier expression of y genes, higher virus yields, and better spread of virus from the site of inoculation. In addition, screens of numerous tumor cell lines for their ability to support the replication of γ₁34.5 mutants of HSV-1 revealed that tumor cell lines vary over a 100-fold range with respect to their ability to support the replication of these virus mutants. In particular, viral yields of HSV R3616 (γ₁34.5) ranged from a low of about 2×10⁴ pfu/ml in PC-3 cells to a high of about 10⁷ pfu/ml in Hep3B cells. The cultures were exposed to 0.1 PFU of virus per cell. Consistent with the present disclosure, the PC-3 cell line was shown to contain active PKR and phosphorylated eIF-2a proteins. The outstanding difference between tumor lines that supported viral replication and those that did not was the state of protein kinase R (PKR). In susceptible cell lines, PKR was not activated whereas in the resistant lines PKR was activated, leading to phosphorylated eIF-2α. Experiments also revealed an inverse correlation between the status of MEK kinase and PKR activation. Importantly, a tumor cell line transformed with a constitutively active mutant of MEK kinase yielded higher titers of γ₁34.5 mutant than the parental cell line or a cell line transformed with a dominant-negative form of MEK kinase. The remaining problem was having MEK expressed in healthy cells, thereby supporting HSV replication and cytolysis. To solve this problem, a way needed to be found to preclude MEK from being expressed in healthy tissues and to restrict its expression to tumor cells. The solution involved construction of a virus in which both copies of the γ₁34.5 gene were replaced by a constitutively active MEK kinase gene driven by a radioinducible promoter, i.e., the HSV-1 gC promoter. Copies of the luciferase gene driven by the immediate-early CMV promoter (pCMV) were also incorporated in these regions of HSV. The rationale for the design is that in normal cells, PKR would be activated and the gC promoter-driven MEK gene would not be expressed. In tumor cells subjected to IR, the gC promoter would be activated, express the constitutively active MEK kinase, and this in turn would block activation of PKR. The expression of the luciferase gene would signal the extent of viral gene expression independent of exposure to IR.

The results of exposing the above-described HSV to tumor xenografts is that a virus encoding a constitutively active MEK kinase driven by an HSV gC promoter, in combination with IR, blocked the growth of a tumor induced by the most resistant tumor cell line identified to date. Virus alone or IR alone was not able to block the growth of the tumors, as described in greater detail below.

The materials and methods used in the experiments described in this Example are described in Example 1, above. In brief, the PC-3 tumor cells used to develop xenografts in mice were cultured in DMEM Nutrient Mixture F. The HSV R2660 was constructed in two steps using conventional recombinant engineering techniques. Recombinants were screened using PCR for the desired construct. Subcutaneous injection of PC-3 cells into mouse hindlimbs was used to develop tumor xenografts, which were monitored for tumor volume. Subsequent to tumor development, mouse hindlimbs were allowed to go untreated, or were treated with either x-radiation or a herpesvirus according to the disclosure, or both x-radiation and a herpesvirus according to the disclosure. Subsequently, tumor volumes were measured and the results are presented in FIG. 17.

The results presented in FIG. 17 shows that in untreated mice, the tumor volume increased 4.5 fold in 15 days. Tumor volume increased, albeit at a reduced rate, in mice treated with virus alone or IR alone. The tumor volume did not increase following administration of both virus and IR. The p value for virus+IR compared to IR alone on Day 7 (first-day p value is less than 0.05) is 0.0394 for 2-tailed, 0.0197 for 1-tailed.

There are several important advantages attending use of a herpesvirus containing an inducible expression control element (e.g., a radioinducible promoter such as pgC) controlling expression of HSV-borne MEK in cancer therapy compared to virus alone or radiotherapy alone. The first advantage is that spatial and temporal control of viral replication and oncolysis can be obtained in that viral replication can be confined principally to the tumor bed because the technology of radiation delivery enables a high tumor tissue-to-normal tissue ratio of radiation delivery. The second advantage is that in clinical situations where the number of installations of virus is limited by location, e.g., brain tumors, repeated doses of radiation may re-induce viral replication as the viral titer decreases following the first burst of radiation-induced viral replication A third advantage is that the activation of MEK in tumors that do not express high titers of MEK allows for a broader use of herpesvirus-based (e.g., HSV-1) therapies since a virus which does not encode MEK would not robustly replicate in a tumor that lacks sufficient MEK activity. The use of the therapeutic herpesvirus constructs disclosed herein, such as gC MEK HSV-1, enables “personalized cancer therapy” because the tumor can be assayed and an appropriate choice of viral vector can be specifically employed depending on the tumor genotype. A fourth advantage is that tumor cells that are radio-resistant are not necessarily resistant to viral oncolysis and cells that are resistant to the virus, either because of intrinsic cellular mechanisms or because they are not reached by the virus, are not necessarily radio-resistant. Therefore, it is expected that the combination therapy involving herpesvirus and IR, particularly suited to the use of radioinducible expression control of virus-borne MEK, will lead to at least an additive effect, and perhaps a synergistic effect, on tumor stasis and/or destruction based on the different mechanisms of tumor cell killing as well as on the genotype-specific spatial and temporal control of gene therapy.

Radioinducible expression control has been exemplified herein using the gC promoter as a radiation-inducible promoter. Various forms of radiation may be used alone or in combination to effect the induction, including but not limited to protons, neutrons, radioisotopes (α, β and γ emitters) and ultraviolet radiation. It is known (see Mezhir et al., Cancer Res. 65:9479-9484 (2005)) that other herpesvirus late gene promoters (e.g., the promoter for US11) are also inducible. The HSV genome has a large number of late genes whose promoters would be suitable for radiation-induced activation of MEK or of other genes in the MEK pathway useful in ensuring a MEK⁺ phenotype in tumor cells. A list of late genes has been published (Roizman et al., “The replication of Herpes simplex viruses” In Fields' Virology, 5th Edition, Knipe, Howley, Griffin, Lamb, Martin, Roizman, and Straus, Editors, Lippincott-Williams and Wilkins, New York, N.Y., pp. 2501-2601 (2007)). Further, a variety of other radioinducible promoters are contemplated for use in the herpesvirus constructs according to the disclosure. For example, the radioinducible promoter may be an Egr-1 promoter, a c-JUN promoter, a TNF-α promoter, an MDR I promoter, a tPA promoter, a recA promoter, a p21 (WAF1) promoter, a CMVIE promoter, an SV40 promoter, a pE9 promoter, a survivin promoter, an IEX-1 promoter and a PKC promoter.

The expression control element, such as the above-described radioinducible promoter, is operatively linked to a coding region for a polypeptide of the MEK pathway. Such polypeptides include, but are not limited to, polypeptide in the MEK pathway is selected from the group consisting of MEK1, MEK2, ERK1, ERK2, Raf-1, A-Raf, B-Raf, mos, Tp1-2, K-Ras, H-Ras and N-Ras. In addition, nucleic acids encoding variants of these polypeptides are contemplated for inclusion in the constructs according to the disclosure. Exemplary variants include, but are not limited to, K-Ras V12, K-Ras D12, K-Ras G12, H-Ras V12, K-Ras D13, N-Ras V12, Raf S338A, Raf S339A, B-Raf V600E, Raf-CAAX, Raf BXB, ΔN3MKK1 S218E/S222D, ΔN3MKK2 S218E/S222D, ERK2 E58Q, ERK2 D122A, ERK2 S151A, ERK2 S221A, ERK2 S151D ERK L73P and a full-length MEK-ERK fusion. Alternative expression control elements, e.g., promoters, are inducible by chemotherapeutic agents, that include, but are not limited to, (a) an alkylating agent, such as a nitrogen mustard (e.g., mechlorethamine, cylophosphamide, ifosfamide, melphalan, chlorambucil), ethylenimine or a methylmelamine (e.g., hexamethylmelamine, thiotepa), an alkyl sulfonate (e.g., busulfan), a nitrosourea (e.g., carmustine, lomustine, chlorozoticin, streptozocin) or a triazine (e.g., dicarbazine); (b) an antimetabolite, such as a folic acid analog (e.g., methotrexate), a pyrimidine analog (e.g., 5-fluorouracil, floxuridine, cytarabine, azauridine) as well as a purine analog or a related compound (e.g., 6-mercaptopurine, 6-thioguanine, pentostatin); (c) a natural product, such as a vinca alkaloid (e.g., vinblastine, vincristine), an epipodophylotoxin (e.g., etoposide, teniposide), an antibiotic (e.g., dactinomycin, daunorubicin, doxorubicin, bleomycin, plicamycin and mitoxanthrone), an enzyme (e.g., L-asparaginase), or a biological response modifier (e.g., Interferon-c0; or (d) a miscellaneous agent, such as a platinum coordination complex (e.g., cisplatin, carboplatin), a substituted urea (e.g., hydroxyurea), a methylhydiazine derivative (e.g., procarbazine), or an adreocortical suppressant (e.g., taxol and mitotane). In some embodiments, cisplatin is a particularly suitable chemotherapeutic agent.

A number of locations within the herpesvirus genome would be suitable for introduction of the coding region for a polypeptide of the MEK pathway. An exemplary location is the locus for the γ₁34.5 gene(s) which, by substituting a coding region for a MEK pathway polypeptide for the γ₁34.5 gene(s) will accomplish both the introduction of an expressible MEK pathway coding region and the loss of expressible γ₁34.5 gene(s), thereby attenuating the virus and providing the virus with the capacity to complete the lytic cycle in MEK⁻ tumor cells with minimal recombinant engineering. Of course, a variety of other mutations, within the γ₁34.5 coding region or affecting γ₁34.5 expression control, are also contemplated, particularly when the expression-controlled coding region for a MEK pathway is not placed into the γ₁34.5 locus of herpesvirus.

Diseases, disorders and conditions amenable to treatment using at least one method according to the disclosure include any form of cancer, such as cancer of the breast, lung, prostate, bladder, colorectal, liver, pancreas, kidney (renal), and head-and-neck; as well as adenoma; cholangioma; cholesteatoma; cyclindroma; cystadenocarcinoma; cystadenoma; granulosa cell tumor; gynandroblastoma; hepatoma; hidradenoma; islet cell tumor; Leydig cell tumor; papilloma; sertoli cell tumor; theca cell tumor; leimyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin. The types of cancers that may be treated also include, but are not limited to, angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia. The disclosure further provides compositions and methods useful in the treatment of other conditions in which cells have become immortalized or hyperproliferative, such as rheumatoid arthritis and macular degeneration.

### REFERENCES

1. Anderson, M. J., G. Casey, C. L. Fasching, and E. J. Stanbridge. 1994. Evidence that wild-type TP53, and not genes on either chromosome 1 or 11, controls the tumorigenic phenotype of the human fibrosarcoma HT1080. Genes Chromosomes Cancer 9:266-81.
2. Andreansky, S., L. Soroceanu, E. R. Flotte, J. Chou, J. M. Markert, G. Y. Gillespie, B. Roizman, and R. J. Whitley. 1997. Evaluation of genetically engineered herpes simplex viruses as oncolytic agents for human malignant brain tumors. Cancer Res 57:1502-9.
3. Ballif, B. A., and J. Blenis. 2001. Molecular mechanisms mediating mammalian mitogen-activated protein kinase (MAPK) kinase (MEK)-MAPK cell survival signals. Cell Growth Differ 12:397-408.
4. Barber, G. N., R. Jagus, E. F. Meurs, A. G. Hovanessian, and M. G. Katze. 1995. Molecular mechanisms responsible for malignant transformation by regulatory and catalytic domain variants of the interferon-induced enzyme RNA-dependent protein kinase, J. Biol. Chem. 270:17423-17428.
5. Barber, G. N., M. Wambach, S. Thompson, R. Jagus, and M. G. Katze. 1995. Mutants of the RNA-dependent protein kinase (PKR) lacking double-stranded RNA binding domain I can act as transdominant inhibitors and induce malignant transformation. Mol Cell Biol 15:3138-46.
6. Bennett, J. J., K. A. Delman, B. M. Burt, A. Mariotti, S. Malhotra, J. Zager, H Petrowsky, S. Mastorides, H. Federoff, and Y. Fong. 2002. Comparison of safety, delivery, and efficacy of two oncolytic herpes viruses (G207 and NV 1020) for peritoneal cancer. Cancer Gene Ther 9:935-45.
7. Cassady, K. A., M. Gross, and B. Roizman. 1998. The herpes simplex virus US11 protein effectively compensates for the gammal(34.5) gene if present before activation of protein kinase R by precluding its phosphorylation and that of the alpha subunit of eukaryotic translation initiation factor 2. J Virol 72:8620-6.
8. Chambers, R., G. Y. Gillespie, L. Soroceanu, S. Andreansky, S. Chatterjee, J. Chou, B. Roizman, and R. J. Whitley. 1995. Comparison of genetically engineered herpes simplex viruses for the treatment of brain tumors in a scid mouse model of human malignant glioma. Proc Nat! Acad Sci (USA) 92:1411-5.
9. Chee, A. V., and B. Roizman. 2004. Herpes simplex virus 1 gene products occlude the interferon signaling pathway at multiple sites. J Virol 78:4185-96.
10. Cheng, G., M. E. Brett, and B. He. 2001. Va1193 and Phe195 of the gamma 1 34.5 protein of herpes simplex virus 1 are required for viral resistance to interferon alpha/beta. Virology 290:115-20.
11. Chou, J., E. R. Kern, R. J. Whitley, and B. Roizman. 1990. Mapping of herpes simplex virus-1 neurovirulence to gamma 134.5, a gene nonessential for growth in culture. Science 250:1262-6.
12. Chou, J., A. P. Poon, J. Johnson, and B. Roizman. 1994. Differential response of human cells to deletions and stop codons in the gamma(1)34.5 gene of herpes simplex virus. J Virol 68:8304-11.
13. Chou, J., and B. Roizman. 1992. The gamma 1(34.5) gene of herpes simplex virus 1 precludes neuroblastoma cells from triggering total shutoff of protein synthesis characteristic of programmed cell death in neuronal cells. Proc Natl Acad Sci (USA) 89:3266-70.
14. Chou, J., and B. Roizman. 1994. Herpes simplex virus 1 gamma(1)34.5 gene function, which blocks the host response to infection, maps in the homologous domain of the genes expressed during growth arrest and DNA damage. Proc Natl Acad Sci (USA) 91:5247-51.
15. Chung, S. M., S. J. Advani, J. D. Bradley, Y. Kataoka, K. Vashistha, S. Y. Yan, J M. Markert, G. Y. Gillespie, R. J. Whitley, B. Roizman, and R. R. Weichselbaum. 2002. The use of a genetically engineered herpes simplex virus (R7020) with ionizing radiation for experimental hepatoma. Gene Ther 9:75-80.
16. Clemens, M. J. 2004. Targets and mechanisms for the regulation of translation in malignant transformation. Oncogene 23:3180-8.
17. Davies, H., G. R. Bignell, C. Cox, P. Stephens, S. Edkins, S. Clegg, J. Teague, H Woffendin, M. J. Garnett, W. Bottomley, N. Davis, E. Dicks, R. Ewing, Y. Floyd, K Gray, S. Hall, R. Hawes, J. Hughes, V. Kosmidou, A. Menzies, C. Mould, A. Parker, C. Stevens, S. Watt, S. Hooper, R. Wilson, H. Jayatilake, B. A. Gusterson, C Cooper, J. Shipley, D. Hargrave, K. Pritchard-Jones, N. Maitland, G. Chenevix-Trench, G. J. Riggins, D. D. Bigner, G. Palmieri, A. Cossu, A. Flanagan, A Nicholson, J. W. Ho, S. Y. Leung, S. T. Yuen, B. L. Weber, H. F. Seigler, T. L Darrow, H. Paterson, R. Marais, C. J. Marshall, R. Wooster, M. R. Stratton, and P A. Futreal. 2002. Mutations of the BRAF gene in human cancer. Nature 417:949-54.
18. Ejercito, P. M., E. D. Kieff, and B. Roizman. 1968. Characterization of herpes simplex virus strains differing in their effects on social behaviour of infected cells. J Gen Virol 2:357-64.
19. Farassati, F., A. D. Yang, and P. W. Lee. 2001. Oncogenes in Ras signalling pathway dictate host-cell permissiveness to herpes simplex virus 1. Nat Cell Biol 3:745-50.
20. Gale, M., Jr., and M. G. Katze. 1998. Molecular mechanisms of interferon resistance mediated by viral-directed inhibition of PKR, the interferon-induced protein kinase Pharmacol Ther 78:29-46.
21. Gupta, S., R. Plattner, C. J. Der, and E. J. Stanbridge. 2000. Dissection of Ras dependent signaling pathways controlling aggressive tumor growth of human fibrosarcoma cells: evidence for a potential novel pathway. Mol Cell Biol 20:9294-306.
22. Gupta, S., and E. J. Stanbridge. 2001. Paired human fibrosarcoma cell lines that possess or lack endogenous mutant N-ras alleles as experimental model for Ras signaling pathways. Methods Enzymol 333:290-306.
23. Hahn, W. C., C. M. Counter, A. S. Lundberg, R. L. Beijersbergen, M. W. Brooks, and R. A. Weinberg. 1999. Creation of human tumour cells with defined genetic elements. Nature 400:464-8.
24. Hallahan, D. E., H. J. Mauceri, L. P. Seung, E. J. Dunphy, J. D. Wayne, N. N. Hanna, A. Toledano, S. Hellman, D. W. Kufe, and R. R. Weichselbaum. 1995. Spatial and temporal control of gene therapy using ionizing radiation. Nat Med 1:786-91.
25. He, B., M. Gross, and B. Roizman. 1997. The gamma(1)34.5 protein of herpes simplex virus 1 complexes with protein phosphatase 1 alpha to dephosphorylate the alpha subunit of the eukaryotic translation initiation factor 2 and preclude the shutoff of protein synthesis by double-stranded RNA-activated protein kinase. Proc Natl Acad Sci (USA) 94:843-8.
26. Hoshino, R., Y. Chatani, T. Yamori, T. Tsuruo, H. Oka, O. Yoshida, Y. Shimada, S.

Ari-i, H. Wada, J. Fujimoto, and M. Kohno. 1999. Constitutive activation of the 41-/43-kDa mitogen-activated protein kinase signaling pathway in human tumors Oncogene 18:813-22.
27. Huang, W., and R. L. Erikson. 1994. Constitutive activation of Mek1 by mutation of serine phosphorylation sites. Proc Natl Acad Sci (USA) 91:8960-3.
28. Ito, T., R. Jagus, and W. S. May. 1994. Interleukin 3 stimulates protein synthesis by regulating double-stranded RNA-dependent protein kinase. Proc Natl Acad Sci (USA) 91:7455-9.
29. Jacquemont, B., and B. Roizman. 1975. RNA synthesis in cells infected with herpes simplex virus. X. Properties of viral symmetric transcripts and of double-stranded RNA prepared from them. J Virol 15:707-13.
30. Katze, M. G. 1995. Regulation of the interferon-induced PKR: can viruses cope? Trends Microbiol 3:75-8.
31. Kozak, M., and B. Roizman. 1975. RNA synthesis in cells infected with herpes simplex virus. IX. Evidence for accumulation of abundant symmetric transcripts in nuclei. J Virol 15:36-40.
32. Le Gall, M., J. C. Chambard, J. P. Breittmayer, D. Grall, J. Pouyssegur, and E. Van Obberghen-Schilling. 2000. The p42/p44 MAP kinase pathway prevents apoptosis induced by anchorage and serum removal. Mol Biol Cell 11:1103-12.
33. Leib, D. A., M. A. Machalek, B. R. Williams, R. H. Silverman, and H. W. Virgin 2000. Specific phenotypic restoration of an attenuated virus by knockout of a host resistance gene. Proc Natl Acad Sci (USA) 97:6097-101.
34. Mansour, S. J., J. M. Candia, J. E. Matsuura, M. C. Maiming, and N. G. Ahn. 1996. Interdependent domains controlling the enzymatic activity of mitogen-activated protein kinase kinase 1. Biochemistry 35:15529-36.
35. Markert, J. M., M. D. Medlock, S. D. Rabkin, G. Y. Gillespie, T. Todo, W. D Hunter, C. A. Palmer, F. Feigenbaum, C. Tornatore, F. Tufaro, and R. L. Martuza. 2000. Conditionally replicating herpes simplex virus mutant, G207 for the treatment of malignant glioma: results of a phase I trial. Gene Ther 7:867-74.
36. Meurs, E. F., J. Galabru, G. N. Barber, M. G. Katze, and A. G. Hovanessian. 1993. Tumor suppressor function of the interferon-induced double-stranded RNA activated protein kinase. Proc Natl Acad Sci (USA) 90:232-6.
38. Nakamura, H., H. Kasuya, J. T. Mullen, S. S. Yoon, T. M. Pawlik, S Chandrasekhar, J. M. Donahue, E. A. Chiocca, R. Y. Chung, and K. K. Tanabe. 2002. Regulation of herpes simplex virus gamma(1)34.5 expression and oncolysis of diffuse liver metastases by Myb34.5. J Clin Invest 109:871-82.
39. Perkins, D. J., and G. N. Barber. 2004. Defects in translational regulation mediated by the alpha subunit of eukaryotic initiation factor 2 inhibit antiviral activity and facilitate the malignant transformation of human fibroblasts. Mol Cell Biol 24:2025-40.
40. Plattner, R., M. J. Anderson, K. Y. Sato, C. L. Fasching, C. J. Der, and E. J Stanbridge. 1996. Loss of oncogenic ras expression does not correlate with loss of tumorigenicity in human cells. Proc Natl Acad Sci (USA) 93:6665-70.
41. Pouyssegur, J., V. Volmat, and P. Lenormand. 2002. Fidelity and spatio-temporal control in MAP kinase (ERKs) signalling. Biochem Pharmacol 64:755-63.
42. Rampling, R., G. Cruickshank, V. Papanastassiou, J. Nicoll, D. Hadley, D. Brennan, R. Petty, A. MacLean, J. Harland, E. McKie, R. Mabbs, and M. Brown. 2000. Toxicity evaluation of replication-competent herpes simplex virus (ICP 34.5 null mutant 1716) in patients with recurrent malignant glioma. Gene Ther 7:859-66.
43. Roller, R. J., and B. Roizman. 1990. The herpes simplex virus Us11 open reading frame encodes a sequence-specific RNA-binding protein. J Virol 64:3463-70.
44. Sebolt-Leopold, J. S., and R. Herrera. 2004. Targeting the mitogen-activated protein kinase cascade to treat cancer. Nat Rev Cancer 4:937-47.
45. Sheaffer, A. K., W. W. Hurlbut, J. T. Stevens, M. Bifano, R. K. Hamatake, R. J. Colonno, and D. J. Tenney. 1995. Characterization of monoclonal antibodies recognizing amino- and carboxy-terminal epitopes of the herpes simplex virus UL42 protein. Virus Res 38:305-14.
46. Shimamura, A., B. A. Ballif, S. A. Richards, and J. Blenis. 2000. Rsk1 mediates a MEK-MAP kinase cell survival signal. Curr Biol 10:127-35.
47. von Gise, A., P. Lorenz, C. Wellbrock, B. Hemmings, F. Berberich-Siebelt, U. R Rapp, and J. Troppmair. 2001. Apoptosis suppression by Raf-1 and MEK1 requires MEK- and phosphatidylinositol 3-kinase-dependent signals. Mol Cell Biol 21:2324-36.
48. Williams, B. R. 2001. Signal integration via PKR. Sci STKE 2001:RE2.
49. Xia, Z., M. Dickens, J. Raingeaud, R. J. Davis, and M. E. Greenberg. 1995. Opposing effects of ERK and JNK-p38 MAP kinases on apoptosis. Science 270:1326-31.
50. Yip-Schneider, M. T., A. Lin, D. Barnard, C. J. Sweeney, and M. S. Marshall. 1999 Lack of elevated MAP kinase (Erk) activity in pancreatic carcinomas despite oncogenic K-ras expression. Int J Oncol 15:271-9.

Numerous modifications and variations of the invention are possible in view of the above teachings and are within the scope of the invention. The entire disclosures of all publications cited herein are hereby incorporated by reference.

What is claimed is:

1. An attenuated herpesvirus expressing less ICP34.5 activity than a wild-type herpesvirus and an inducible expression control element operatively linked to a coding region for a polypeptide in the MEK pathway.

2. The attenuated herpesvirus according to claim 1 wherein the herpesvirus is HSV-1.

3. The attenuated herpesvirus according to claim 1 wherein the herpesvirus lacks a γ₁34.5 gene capable of expressing active ICP34.5.

4. The attenuated herpesvirus according to claim 3 wherein at least 10% of each coding region for ICP34.5 has been deleted.

5. The attenuated herpesvirus according to claim 1 wherein the polypeptide in the MEK pathway is selected from the group consisting of MEK1, MEK2, ERK1, ERK2, Raf-1, A-Raf, B-Raf, mos, Tp1-2, K-Ras, H-Ras and N-Ras.

6. The attenuated herpesvirus according to claim 5 wherein the polypeptide is MEK-1 or MEK-2.

7. The attenuated herpesvirus according to claim 1 wherein the polypeptide in the MEK pathway is selected from the group consisting of K-Ras V12, K-Ras D12, K-Ras G12, H-Ras V12, K-Ras D13, N-Ras V12, Raf S338A, Raf S339A, B-Raf V600E, Raf-CAAX, Raf BXB, ΔN3MKK1 S218E/S222D, ΔN3MKK2 S218E/S222D, ERK2 E58Q, ERK2 D122A, ERK2 S151A, ERK2 S221A, ERK2 S151D ERK L73P and a full-length MEK-ERK fusion.

8. The attenuated herpesvirus according to claim 1 wherein the inducible expression control element is inducible by radiation or by exposure to a chemotherapeutic agent.

9. The attenuated herpesvirus according to claim 8 wherein the inducible expression control element is a radioinducible promoter.

10. The attenuated herpesvirus according to claim 9 wherein the radioinducible promoter is selected from the group consisting of an Egr-1 promoter, a c-JUN promoter, a TNF-α promoter, an MDR1 promoter, a tPA promoter, a recA promoter, a p21 (WAF1) promoter, a CMVIE promoter, an SV40 promoter, a pE9 promoter, a survivin promoter, an IEX-1 promoter and a PKC promoter.

11. The attenuated herpesvirus according to claim 9 wherein the radioinducible promoter is the promoter for HSV gC.

12. The attenuated herpesvirus according to claim 8 wherein the chemotherapeutic agent is selected from the group consisting of a nitrogen mustard, an ethylenimine, a methylmelamine, an alkyl sulfonate, a nitrosourea, a triazine, a folic acid analog, a pyrimidine analog, a purine analog, a vinca alkaloid, an epipodophylotoxin, an antibiotic, an enzyme, a biological response modifier, a platinum coordination complex, a substituted urea, a methylhydiazine derivative and an adreocortical suppressant.

13. The attenuated herpesvirus according to claim 8 wherein the chemotherapeutic agent is selected from the group consisting of mechlorethamine, cylophosphamide, ifosfamide, melphalan, chlorambucil, hexamethylmelamine, thiotepa, busulfan, carmustine, iomustine, chlorozoticin, streptozocin, dicarbazine, methotrexate, 5-fluorouracil, floxuridine, cytarabine, azauridine, 6-mercaptopurine, 6-thioguanine, pentostatin, vinblastine, vincristine, etoposide, teniposide, dactinomycin, daunorubicin, doxorubicin, bleomycin, plicamycin, mitoxanthrone, L-asparaginase, Interferon-c0, cisplatin, carboplatin, hydroxyurea, procarbazine, taxol and mitotane.

14. The attenuated herpesvirus according to claim 1 further comprising a coding region for an expressible marker.

15. A method of treating a cell proliferative disorder comprising administering a therapeutically effective amount of a herpesvirus according to claim 1 in combination with a therapeutically effective amount of an anti-cell proliferation agent selected from the group consisting of radiation and a chemotherapeutic agent.

16. The method according to claim 15 wherein radiation is selected from the group consisting of a proton emission, a neutron emissioll, an α radioisotope, a β radioisotope, a γ radioisotope and ultraviolet radiation.

17. The method according to claim 16 wherein radiation comprises ionizing radiation.

18. The method according to claim 15 wherein the disorder is selected from the group consisting of a cancer, rheumatoid arthritis and macular degeneration.

19. A method of ameliorating a symptom of a cell proliferative disorder comprising administering a therapeutically effective amount of a herpesvirus according to claim 1 in combination with a therapeutically effective amount of an anti-cell proliferation agent selected from the group consisting of radiation and a chemotherapeutic agent.

20. Use of the herpesvirus according to claim 1 in the preparation of a medicament for the treatment of a subject with a cell proliferation disorder.

21. A composition comprising the herpesvirus according to claim 1 in combination with a pharmaceutically acceptable adjuvant, carrier or diluent.


SEQUENCE LISTING (24 sequences)

SEQ ID NO: 1 - DNA, 2222 bp, Homo sapiens
attcggcacgagggaggaagcgagaggtgctgccctccccccggagttggaagcgcgttacccgggtccaaaatgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaagtgtttgggaagcaacaaagagcgagtcccctgcccggtggtttgccatgtcgcttttgggcctccttcccatgcctgtctctgttcagatgtgcatttcacctgtgacaaaggatgaagaacacagcatgtgccaagattctactcttgtcatttttaatattactgtctttattcttattactattattgttcccctaagtggattggctttgtgcttggggctatttgtgtgtatgctgatgatcaaaacctgtgccaggctgaattacagtgaaatttttggtgaatgtgggtagtcattcttacaattgcactgctgttcctgctccatgactggctgtctgcctgtattttcggactttgacatttgacatttggtggactttatcttgctgggcatactttctctctaggagggagccttgtgagatccttcacaggcagtgcatgtgaagcatgctttgctgctatgaaaatgagcatcagagagtgtacatcatgttattttattattattatttgcttttcatgtagaactcagcagttgacatccaaatctagccagagcccttcactgccatgatagctggggcttcaccagtctgtctactgtggtgatctgtagacttctggttgtatttctatatttattttcagtatactgtgtgggatacttagtggtatgtctctttaagttttgattaatgtttcttaaatggaattatttgaatgtcacaaattgatcaagatattaaaatgtcggatttatctttccccatatccaagtaccaatgctgttgtaaacaacgtgtatagtgcctaaaattgtatgaaaatccttttaaccattttaacctagatgtttaacaaatctaatctcttattctaataaatatactatgaaataaaaaaaaaaggagaaagctaaaaaaaaaaaaaaaaaaaaaaaaaaaaaaaaaaaaaaa

SEQ ID NO: 2 - protein, 393 aa, Homo sapiens [sequence omitted]

SEQ ID NO: 3 - DNA, 1611 bp, Homo sapiens
acataatttctggagccctgtaccaacgtgtggccacatattctgtcaggaaccctgtgtgatcatggtctggatctgcaacacgggccaggccaaagtcacagatcttgagatcacaggtggtgttgagcagcaggcaggcaggcaatcggtccgagtggctgtcggctcttcagctctccgctcggcgtcttccttcctctcccggtcagcgtcggcggctgcaccggcggcgggcagtcctgcgggaggggcgacaagagctgaggcgcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttccaacaggcccatctttccagggaagcattatcttgaccagctgaatcacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctaaaagaactaatttttgaagagactgctagattccagccaggatacagatcttaaatttgtcaggacaagggctcagaggactggacgtgctcagacatcggtgttcttcttcccagttcttgacccctggtcctgtctccagcccgtcttggcttatccactttgactcctttgagccgtttggaggggcggtttctggtagttgtggcttttatgctttcaaagaatttcttcagtccagagaattcactggcc

SEQ ID NO: 4 - protein, 360 aa, Homo sapiens [sequence omitted]

SEQ ID NO: 5 - DNA, 1759 bp, Homo sapiens
cccctgcctctcggactcgggctgcggcgtcagccttcttcgggcctcggcagcggtagcggctcgctcgcctcagccccagcgcccctcggctaccctcggcccaggcccgcagcgccgcccgccctcggccgccccgacgccggcctgggccgcggccgcagccccgggctcgcgtaggcgccgaccgctcccggcccgccccctatgggccccggctagaggcgccgccgccgccggcccgcggagccccgatgctggcccggaggaagccggtgctgccggcgctcaccatcaaccctaccatcgccgagggcccatcccctaccagcgagggcgcctccgaggcaaacctggtggacctgcagaagaagctggaggagctggaacttgacgagcagcagaagaagcggctggaagcctttctcacccagaaagccaaggtcggcgaactcaaagacgatgacttcgaaaggatctcagagctgggcgcgggcaacggcggggtggtcaccaaagtccagcacagaccctcgggcctcatcatggccaggaagctgatccaccttgagatcaagccggccatccggaaccagatcatccgcgagctgcaggtcctgcacgaatgcaactcgccgtacatcgtgggcttctacggggccttctacagtgacggggagatcagcatttgcatggaacacatggacggcggctccctggaccaggtgctgaaagaggccaagaggattcccgaggagatcctggggaaagtcagcatcgcggttctccggggcttggcgtacctccgagagaagcaccagatcatgcaccgagatgtgaagccctccaacatcctcgtgaactctagaggggagatcaagctgtgtgacttcggggtgagcggccagctcatagactccatggccaactccttcgtgggcacgcgctcctacatggctccggagcggttgcagggcacacattactcggtgcagtcggacatctggagcatgggcctgtccctggtggagctggccgtcggaaggtaccccatccccccgcccgacgccaaagagctggaggccatctttggccggcccgtggtcgacggggaagaaggagagcctcacagcatctcgcctcggccgaggccccccgggcgccccgtcagcggtcacgggatggatagccggcctgccatggccatctttgaactcctggactatattgtgaacgagccacctcctaagctgcccaacggtgtgttcacccccgacttccaggagtttgtcaataaatgcctcatcaagaacccagcggagcgggcggacctgaagatgctcacaaaccacaccttcatcaagcggtccgaggtggaagaagtggattttgccggctggttgtgtaaaaccctgcggctgaaccagcccggcacacccacgcgcaccgccgtgtgacagtggccgggctccctgcgtcccgctggtgacctgcccaccgtccctgtccatgccccgcccttccagctgaggacaggctggcgcctccacccaccctcctgcctcacccctgcggagagcaccgtggcggggcgacagcgcatgcaggaacgggggtctcctctcctgcccgtcctggccggggtgcctctggggacgggcgacgctgctgtgtgtggtctcagaggctctgcttccttaggttacaaaacaaaacagggagagaaaaagcaaaaaaaaaaaaaaaaaaaaaaaaaaaa

SEQ ID NO: 6 - protein, 400 aa, Homo sapiens [sequence omitted]

SEQ ID NO: 7 - DNA, 1963 bp, Homo sapiens
gaaacgtcccgtgtgggaggggcgggtctgggtgcggctgccgcatgactcgtggttcggaggcccacgtggccggggcggggactcaggcgcctggcagccgactgattacgtagcgggcggggccggaagtgccgctccttggtgggggctgttcatggcggttccggggtctccaacatttttcccggtctgtggtcctaaatctgtccaaagcagaggcagtggagcttgaggttcttgctggtgtgaaatgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaacaagatacttttaaagttttgtcagaaaagagccactttcaagctgcactgacaccctggtcctgacttcctggaggagaagtattcctgttgctgtcttcagtctcacagagaagctcctgctacttccccagctctcagtagtttagtacaataatctctatttgagaagttctcagaataactacctcctcacttggctgtctgaccagagaatgcacctcttgttactccctgttatttttctgccctgggttcttccacagcacaaacacacctcaacacacctctgccaccccaggtttttcatctgaaaagcagttcatgtctgaaacagagaaccaaaccgcaaacgtgaaattctattgaaaacagtgtcttgagctctaaagtagcaactgctggtgattttttttttctttttactgttgaacttagaactatgcctaatttttggagaaatgtcataaattactgttttgccaagaatatagttattattgctgtttggtttgtttataatgttatcggctctattctctaaactggcatctgctctagattcataaatacaaaaatgaatactgaattttgagtctatcctagtcttcacaactttgacgtaattaaatccaacttttcacagtgaagtgcctttttcctagaagtggtttgtagactcctttataatatttcagtggaatagatgtctcaaaaatccttatgcatgaaatgaatgtctgagatacgtctgtgacttatctaccattgaaggaaagctatatctatttgagagcagatgccattttgtacatgtatgaaattggttttccagaggcctgttttggggctttcccaggagaaagatgaaactgaaagcatatgaataatttcacttaataatttttacctaatctccacttttttcataggttactacctatacaatgtatgtaatttgtttcccctagcttactgataaacctaatattcaatgaacttccatttgtattcaaatttgtgtcataccagaaagctctacatttgcagatgttcaaatattgtaaaactttggtgcattgttatttaatagctgtgatcagtgattttcaaacctcaaatatagtatattaacaaatt

SEQ ID NO: 8 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 9 - DNA, 5775 bp, Homo sapiens
tcctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagcttgtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctcgaagtgccagtattcccagagttttggtttttgaactagcaatgcctgtgaaaaagaaactgaatacctaagatttctgtcttggggtttttggtgcatgcagttgattacttcttatttttcttaccaagtgtgaatgttggtgtgaaacaaattaatgaagcttttgaatcatccctattctgtgttttatctagtcacataaatggattaattactaatttcagttgagaccttctaattggtttttactgaaacattgagggacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagctttttcctgttaaggcagacccagtatgaatgggattattatagcaaccattttggggctatatttacatgctactaaatttttataataattgaaaagattttaacaagtataaaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaagccaggccctgtgtgaaccttgagctttcatagagagtttcacagcatggactgtgtgccccacggtcatccgagtggttgtacgatgcattggttagtcaaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggttgttggtgacttaggttctagataggtgtcttttaggactctgattttgaggacatcacttactatccatttcttcatgttaaaagaagtcatctcaaactcttagttttttttttttacactatgtgatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttattttcctcagggctcaagagaatctgacagataccataaagggatttgacctaatcactaattttcaggtggtggctgatgctttgaacatctctttgctgcccaatccattagcgacagtaggatttttcaaccctggtatgaatagacagaaccctatccagtggaaggagaatttaataaagatagtgcagaaagaattccttaggtaatctataactaggactactcctggtaacagtaatacattccattgttttagtaaccagaaatcttcatgcaatgaaaaatactttaattcatgaagcttacttttttttttttggtgtcagagtctcgctcttgtcacccaggctggaatgcagtggcgccatctcagctcactgcaaccttccatcttcccaggttcaagcgattctcgtgcctcggcctcctgagtagctgggattacaggcgtgtgcactacactcaactaatttttgtatttttaggagagacggggtttcacctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtactgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacagtgcctgaactagttcacagacaagggaaacttctatgtaaaaatcactatgatttctgaattgctatgtgaaactacagatctttggaacactgtttaggtagggtgttaagacttgacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagactaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgatttttttttttcttctaaacattttttcttcaaaacagtatatataactttttttaggggattttttttagacagcaaaaaactatctgaagatttccatttgtcaaaaagtaatgatttcttgataattgtgtagtgaatgttttttagaacccagcagttaccttgaaagctgaatttatatttagtaacttctgtgttaatactggatagcatgaattctgcattgagaaactgaatagctgtcataaaatgctttctttcctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagttatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggcctgtcttttaactatttttgtatagtgtaaactgaaacatgcacattttgtacattgtgctttcttttgtgggtcatatgcagtgtgatccagttgttttccatcatttggttgcgctgacctaggaatgttggtcatatcaaacattaaaaatgaccactcttttaatgaaattaacttttaaatgtttataggagtatgtgctgtgaagtgatctaaaatttgtaatatttttgtcatgaactgtactactcctaattattgtaatgtaataaaaatagttacagtgactatgagtgtgtatttattcatgcaaatttgaactgtttgccccgaaatggatatggatactttataagccatagacactatagtataccagtgaatcttttatgcagcttgttagaagtatccttttattttctaaaaggtgctgtggatattatgtaaaggcgtgtttgcttaaacaattttccatatttagaagtagatgcaaaacaaatctgcctttatgacaaaaaaataggataacattatttatttatttccttttatcaataaggtaattgatacacaacaggtgacttggttttaggcccaaaggtagcagcagcaacattaataatggaaataattgaatagttagttatgtatgttaatgccagtcaccagcaggctatttcaaggtcagaagtaatgactccatacatattatttatttctataactacatttaaatcattaccagg

SEQ ID NO: 10 - protein, 188 aa, Homo sapiens
MTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 11 - DNA, 571 bp, Homo sapiens
catgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 12 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 13 - DNA, 2477 bp, Homo sapiens
cgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaa

SEQ ID NO: 14 - protein, 766 aa, Homo sapiens [sequence omitted]

SEQ ID NO: 15 - DNA, 5916 bp, Homo sapiens
gcccctccctccgcccgcccgccggcccgcccgtcagtctggcaggcaggcaggcaatcggtccgagtggctgtcggctcttcagctctcccgctcggcgtcttccttcctcctcccggtcagcgtcggcggctgcaccggcggcggcgcagtccctgcgggaggggcgacaagagctgagcggcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaaatttgtcaggacaagggctcagaggactggacgtgctcagacatcggtgttcttcttcccagttcttgacccctggtcctgtctccagcccgtcttggcttatccactttgactcctttgagccgtttggaggggcggtttctggtagttgtggcttttatgctttcaaagaatttcttcagtccagagaattcctcctggcagccctgtgtgtgtcacccattggtgacctgcggcagtatgtacttcagtgcacctactgcttactgttgctttagtcactaattgctttctggtttgaaagatgcagtggttcctccctctcctgaatccttttctacatgatgccctgctgaccatgcagccgcaccagagagagattcttccccaattggctctagtcactggcatctcactttatgatagggaaggctactacctagggcactttaagtcagtgacagccccttatttgcacttcaccttttgaccataactgtttccccagagcaggagcttgtggaaataccttggctgatgttgcagcctgcagcaagtgcttccgtctccggaatccttggggagcacttgtccacgtcttttctcatatcatggtagtcactaacatatataaggtatgtgctattggcccagcttttagaaaatgcagtcatttttctaaataaaaaggaagtactgcacccagcagtgtcactctgtagttactgtggtcacttgtaccatatagaggtgtaacacttgtcaagaagcgttatgtgcagtacttaatgtttgtaagacttacaaaaaaagatttaaagtggcagcttcactcgacatttggtgagagaagtacaaaggttgcagtgctgagctgtgggcggtttctggggatgtcccagggtggaactccacatgctggtgcatatacgcccttgagctacttcaaatgtgggtgtttcagtaaccacgttccatgcctgaggatttagcagagaggaacactgcgtctttaaatgagaaagtatacaattctttttccttctacagcatgtcagcatctcaagttcatttttcaacctacagtataacaatttgtaataaagcctccaggagctcatgacgtgaagcactgttctgtcctcaagtactcaaatatttctgatactgctgagtcagactgtcagaaaaagctagcactaactcgtgtttggagctctatccatattttactgatctctttaagtatttgttcctgccactgtgtactgtggagttgactcggtgttctgtcccagtgcggtgcctcctcttgacttccccactgctctctgtggtgagaaatttgccttgttcaataattactgtaccctcgcatgactgttacagctttctgtgcagagatgactgtccaagtgccacatgcctacgattgaaatgaaaactctattgttacctctgagttgtgttccacggaaaatgctatccagcagatcatttaggaaaaataattctatttttagcttttcatttctcagctgtccttttttcttgtttgatttttgacagcaatggagaatgggttatataaagactgcctgctaatatgaacagaaatgcatttgtaattcatgaaaataaatgtacatcttctatcttcacattcatgttaagattcagtgttgctttcctctggatcagcgtgtctgaatggacagtcaggttcaggttgtgctgaacacagaaatgctcacaggcctcactttgccgcccaggcactggcccagcacttggatttacataagatgagttagaaaggtacttctgtagggtcctttttacctctgctcggcagagaatcgatgctgtcatgttcctttattcacaatcttaggtctcaaatattctgtcaaaccctaacaaagaagccccgacatctcaggttggattccctggttctctctaaagagggcctgcccttgtgccccagaggtgctgctgggcacagccaagagttgggaagggccgccccacagtacgcagtcctcaccacccagcccagggtgctcacgctcaccactcctgtggctgaggaaggatagctggctcatcctcggaaaacagacccacatctctattcttgccctgaaatacgcgcttttcacttgcgtgctcagagctgccgtctgaaggtccacacagcattgacgggacacagaaatgtgactgttaccggataacactgattagtcagttttcatttataaaaaagcattgacagttttattactcttgtttctttttaaatggaaagttactattataaggttaatttggagtcctcttctaaatagaaaaccatatccttggctactaacatctggagactgtgagctccttcccattccccttcctggtactgtggagtcagattggcatgaaaccactaacttcattctagaatcattgtagccataagttgtgtgctttttattaatcatgccaaacataatgtaactgggcagagaatggtcctaaccaaggtacctatgaaaagcgctagctatcatgtgtagtagatgcatcattttggctcttcttacatttgtaaaaatgtacagattaggtcatcttaattcatattagtgacacggaacagcacctccactatttgtatgttcaaataagctttcagactaatagcttttttggtgtctaaaatgtaagcaaaaaattcctgctgaaacattccagtcctttcatttagtataaaagaaatactgaacaagccagtgggatggaattgaaagaactaatcatgaggactctgtcctgacacaggtcctcaaagctagcagagatacgcagacattgtggcatctgggtagaagaatactgtattgtgtgtgcagtgcacagtgtgtggtgtgtgcacactcattccttctgctcttgggcacaggcagtgggtgtagaggtaaccagtagctttgagaagctacatgtagctcaccagtggttttctctaaggaatcacaaaagtaaactacccaaccacatgccacgtaatatttcagccattcagaggaaactgttttctctttatttgcttatatgttaatatggtttttaaattggtaacttttatatagtatggtaacagtatgttaatacacacatacatacgcacacatgctttgggtccttccataatacttttatatttgtaaatcaatgttttggagcaatcccaagtttaagggaaatatttttgtaaatgtaatggttttgaaaatctgagcaatccttttgcttatacatttttaaagcatttgtgctttaaaattgttatgctggtgtttgaaacatgatactcctgtggtgcagatgagaagctataacagtgaatatgtggtttctcttacgtcatccaccttgacatgatgggtcagaaacaaatggaaatccagagcaagtcctccagggttgcaccaggtttacctaaagcttgttgccttttcttgtgctgtttatgcgtgtagagcactcaagaaagttctgaaactgctttgtatctgctttgtactgttggtgccttcttggtattgtaccccaaaattctgcatagattatttagtataatggtaagttaaaaaatgttaaaggaagattttattaagaatctgaatgtttattcattatattgttacaatttaacattaacatttatttgtggtatttgtgatttggttaatctgtataaaaattgtaagtagaaaggtttatatttcatcttaattcttttgatgttgtaaacgtactttttaaaagatggattatttgaatgtttatggcacctgacttgtaaaaaaaaaaaactacaaaaaaatccttagaatcattaaattgtgtccctgtattaccaaaataacacagcaccgtgcatgtatagtttaattgcagtttcatctgtgaaaacgtgaaattgtctagtccttcgttatgttccccagatgtcttccagatttgctctgcatgtggtaacttgtgttagggctgtgagctgttcctcgagttgaatggggatgtcagtgctcctagggttctccaggtggttcttcagaccttcacctgtgggggggggggtaggcggtgcccacgcccatctcctcatcctcctgaacttctgcaaccccactgctgggcagacatcctgggcaaccccttttttcagagcaagaagtcataaagataggatttcttggacatttggttcttatcaatattgggcattatgtaatgacttatttacaaaacaaagatactggaaaatgttttggatgtggtgttatggaaagagcacaggccttggacccatccagctgggttcagaactaccccctgcttataactgcggctggctgtgggccagtcattctgcgtctctgctttcttcctctgcttcagactgtcagctgtaaagtggaagcaatattacttgccttgtatatggtaaagattataaaaatacatttcaactgttcagcatagtacttcaaagcaagtactcagtaaatagcaagtctttttaaa

SEQ ID NO: 16 - protein, 360 aa, Homo sapiens [sequence omitted]

SEQ ID NO: 17 - DNA, 1499 bp, Homo sapiens
gcccctccctccgcccgcccgccggcccgcccgtcagtctggcaggcaggcaggcaatcggtccgagtggctgtcggctcttcagctctcccgctcggcgtcttccttcctcctcccggtcagcgtcggcggctgcaccggcggcggcgcagtccctgcgggaggggcgacaagagctgagcggcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaaatttgtcaggtacctggagtttaatacagtgagctctagcaagggaggcgctgccttttgtttctagaatattatgttcctcaaggtccattattttgtattcttttccaagctccttattggaaggtatttttttaaatttagaattaaaaattatttagaaagttacatataaa

SEQ ID NO: 18 - protein, 360 aa, Homo sapiens [sequence omitted]

SEQ ID NO: 19 - DNA, 20 bp, Artificial Sequence
  Synthetic Primer
gagtggttacgcgcggcgcg

SEQ ID NO: 20 - DNA, 22 bp, Homo sapiens
  MISC_FEATURE: MEK Primer
gcagagctggtcccgttaactg

SEQ ID NO: 21 - DNA, 20 bp, Artificial Sequence
  Synthetic Primer
gcactactcgcctctgcacg

SEQ ID NO: 22 - DNA, 22 bp, Artificial Sequence
  Synthetic Primer
cgctgaattggaatccatcttg

SEQ ID NO: 23 - DNA, 19 bp, Artificial Sequence
  Synthetic Primer
ccgcgtttatgaacaaacg

SEQ ID NO: 24 - DNA, 18 bp, Artificial Sequence
  Synthetic Primer
gcagatcttggtggcgtg